(19) United States
(12) Patent Application Publication
(10) Pub. No.: US 2023/0178206 A1
(43) Pub. Date: Jun. 8, 2023

(54) METHOD AND APPARATUS FOR CONCUSSION RECOVERY

(71) Applicant: Neurovine Inc., Ottawa (CA)

(72) Inventors: Locillo PINO, Nepean (CA); Ashleigh KENNEDY, Ottawa (CA); Matthew KENNEDY, Ottawa (CA); Mark CAMPBELL, Ottawa (CA); Marwan ELKHOLY, Ottawa (CA); Matthew BAILEY, Ottawa (CA); Chris EDWARDS, Ottawa (CA); James HENDERSON, Gatineau (CA)

(21) Appl. No.: 17/640,392

(22) PCT Filed: Aug. 27, 2021
(86) PCT No.: PCT/CA2021/051196

§ 371 Date: Mar. 4, 2022

Related U.S. Application Data
(60) Provisional application No. 63/071,431, filed on Aug. 28, 2020.

(51) Int. Cl. G16H 20/30; A61B 5/375; A61B 5/00; A61B 5/291

(52) U.S. Cl. CPC G16H 20/30; A61B 5/375; A61B 5/746; A61B 5/291; A61B 5/0006; A61B 5/6803; A61B 2505/09; A61B 2560/0223

(57) ABSTRACT

A method and an apparatus for generating a brain break alert is provided. The method includes receiving electroencephalographic (EEG) data of a patient engaged in a cognitive activity at a system server from a user device. The EEG data is captured by electrode of an EEG headset, where the electrode is in physical contact with the patient, and the EEG headset is communicably coupled to the user device. Brain energy used (BEU) by the patient is determined based on the EEG data, brain energy available (BEA) to the patient is determined as a function of the BEU and a predefined initial brain energy (BEI) of the patient, where the BEA is proportional to a difference between the BEI and the BEU, and the BEA satisfying a brain break alert threshold is determined, upon which an instruction to generate a brain break alert (BBA) is sent to the user device.

This application is a non-provisional of U.S. Provisional Patent Application Ser. No. 63/071,431, filed on Aug. 28, 2020, which is incorporated herein by reference.

### FIELD

Embodiments of the invention relate to assist patients having a brain injury, and more particularly a method and apparatus for concussion recovery.

### BACKGROUND

Concussions are invisible, in that it is almost impossible to delve deep into the brain of an injured patient to analyze the extent of the injury. Thus, concussion recovery is a complex field. Each concussion is different, and each patient's personal limits are different. For example, light exercise is recommended to improve concussion recovery—yet too much exercise can set recovery back. Furthermore, too much cognitive activity can also set recovery back, triggering depression caused by social isolation and boredom. There are other debilitating effects: concussed patients are prone to anxiety caused by the helplessness of not knowing what to do, as concussion recovery protocol is often vague and poorly-defined. Currently, physicians do not have data to guide patients; instead, a physician's assessment relies on a patient's subjective reports. However, the human brain is a complex instrument that needs scientific analysis and data to manage the recovery process. As such, there is a need for a science-based, real-time data analysis of concussions.

Patients often report that concussion recovery protocols are vague, generic and symptom-based. They feel alone and confused because current recovery processes cause a disruption in their daily lives. Reports indicate that patients either push themselves too hard and suffer recurring symptoms. Or they retreat and have a tumultuous but slow recovery plagued by depression, helplessness, anxiety from social isolation, and guilt from the added burden they are causing their loved ones and co-workers.

Current research has shown that light exercise speeds concussion recovery (Leddy et al. 2019). However, over-exertion is related to a metabolic cascade that causes patients to regress during their concussion recovery. The same research team has disclosed the development of a treadmill test to determine what level of aerobic exercise is appropriate for concussion patients looking to speed their recovery process.

While this can act as a guide to physical activity post-concussion, there are no tools to help concussion patients navigate their cognitive load post-concussion (which is important for getting back to school, work and life in general). Similar to physical exercise, subthreshold cognitive activity is recommended for improved recovery and good mental health (Concussion Consortium 2017). However, too much activity leads to the onset of concussion symptoms and recovery regression (Rose et al. 2016). This pattern pushes concussion patients into a negative cycle called post-concussive syndrome (PCS). Once a patient is in this pattern, they can suffer from symptoms for anywhere between 3 and 12 months, and, occasionally for life.

In fact, the pressure and speed of our society, combined with the invisible nature of concussions, have contributed to a sizeable increase in PCS found across all ages between 2010 and 2015 (BCBS Concussion).

While the International Concussion Consortium recommends a personalized treatment plan to help prevent PCS and navigate the return to school, life, and work, such a protocol for the prevention does not exist yet. The main reason for this is that concussions are invisible. Currently, there is no clear way to quantify this type of brain injury, and no way to scientifically monitor improvement. Furthermore, there is no easy way to measure cognitive load or ‘over-exertion’.

According there exists a need in the art for improved techniques for concussion recovery.

### SUMMARY

Disclosed herein are machine-learning-based systems and methods for concussion recovery. The systems and methods comprise a software platform that uses EEG from a portable headset to create a personalized concussion rehabilitation tool.

In one aspect, a method for generating a brain break alert includes receiving, at a system server from a user device, electroencephalographic (EEG) data of a patient engaged in a cognitive activity, where the EEG data is captured by at least one electrode of an EEG headset, the at least one electrode is in physical contact with the patient, and the EEG headset is communicably coupled to the user device, determining, at the system server, brain energy used (BEU) by the patient based on the EEG data, determining, at the system server, brain energy available (BEA) to the patient as a function of the BEU and a predefined initial brain energy (BEI) of the patient, where the BEA is proportional to a difference between the BEI and the BEU, determining, at the system server, that the BEA has satisfied a brain break alert threshold, and sending, from the system server to the user device, upon the brain break alert threshold being satisfied, an instruction to generate a brain break alert (BBA) on the user device.

In one aspect, a computing apparatus includes a processor. The computing apparatus also includes a memory storing instructions that, when executed by the processor, configure the apparatus to receive, at a system server from a user device, electroencephalographic (EEG) data of a patient engaged in a cognitive activity, where the EEG data is captured by at least one electrode of an EEG headset, the at least one electrode is in physical contact with the patient, and the EEG headset is communicably coupled to the user device, determine, at the system server, brain energy used (BEU) by the patient based on the EEG data, determine, at the system server, brain energy available (BEA) to the patient as a function of the BEU and a predefined initial brain energy (BEI) of the patient, where the BEA is proportional to a difference between the BEI and the BEU, determine, at the system server, that the BEA has satisfied a brain break alert threshold, and send, from the system server to the user device, upon the brain break alert threshold being satisfied, an instruction to generate a brain break alert (BBA) on the user device.

In one aspect, a non-transitory computer-readable storage medium, the computer-readable storage medium including instructions that when executed by a computer, cause the computer to receive, at a system server from a user device, electroencephalographic (EEG) data of a patient engaged in a cognitive activity, where the EEG data is captured by at least one electrode of an EEG headset, the at least one electrode is in physical contact with the patient, and the EEG headset is communicably coupled to the user device, determine, at the system server, brain energy used (BEU) by the patient based on the EEG data, determine, at the system server, brain energy available (BEA) to the patient as a function of the BEU and a predefined initial brain energy (BEI) of the patient, where the BEA is proportional to a difference between the BEI and the BEU, determine, at the system server, that the BEA has satisfied a brain break alert threshold, and send, from the system server to the user device, upon the brain break alert threshold being satisfied, an instruction to generate a brain break alert (BBA) on the user device.

In one aspect, there is provided a method for concussion recovery, the method comprising: receiving, by a processor, training EEG data from a patient; pre-processing, by a processing module, the training EEG data; calibrating, by a calibration module, the training EEG data to provide a first threshold and second threshold, the first threshold being higher than the second threshold; training, by a training module, a machine learning algorithm based on the training EEG data to provide an instantaneous mental workload (IMWL) score; receiving, by the processor, EEG data from the patient; pre-processing, by the processing module, the EEG data; predicting, by the machine learning algorithm, the IMWL score of the EEG data; adjusting, by a brain energy module, a Total Brain Energy Measure if the IMWL score is above the first threshold or below the second threshold; comparing, by the processor, the Total Brain Energy Measure to a third threshold; and raising an alarm, by the processor, if the Total Brain Energy Measure is less than the third threshold.

In another aspect, there is provided a system comprising: a processor; and a memory storing instructions that, when executed by the processor, configure the system to: receive, by the processor, training EEG data from a patient; pre-process, by a processing module, the training EEG data; calibrate, by a calibration module, the training EEG data to provide a first threshold and second threshold, the first threshold being higher than the second threshold; train, by a training module, a machine learning algorithm based on the training EEG data to provide an instantaneous mental workload (IMWL) score; receive, by the processor, EEG data from the patient; pre-process, by the processing module, the EEG data; predict, by the machine learning algorithm, the IMWL score of the EEG data; adjust, by a brain energy module, a Total Brain Energy Measure if the IMWL score is above the first threshold or below the second threshold; compare, by the processor, the Total Brain Energy Measure to a third threshold; and raise an alarm, by the processor, if the Total Brain Energy Measure is less than the third threshold.

In yet another aspect, there is provided a non-transitory computer-readable storage medium, the computer-readable storage medium including instructions that when executed by a computer, cause the computer to: receive, by a processor, training EEG data from a patient; pre-process, by a processing module, the training EEG data; calibrate, by a calibration module, the training EEG data to provide a first threshold and second threshold, the first threshold being higher than the second threshold; train, by a training module, a machine learning algorithm based on the training EEG data to provide an instantaneous mental workload (IMWL) score; receive, by the processor, EEG data from the patient; pre-process, by the processing module, the EEG data; predict, by the machine learning algorithm, the IMWL score of the EEG data; adjust, by a brain energy module, a Total Brain Energy Measure if the IMWL score is above the first threshold or below the second threshold; compare, by the processor, the Total Brain Energy Measure to a third threshold; and raise an alarm, by the processor, if the Total Brain Energy Measure is less than the third threshold.

The systems and methods disclosed herein provide personalized, real-time data on a patient's cognitive load and deliver an indicator when the patient is progressing towards a fatigued brain state, prompting the patient to take a “brain break”. The systems and methods do the same for physical exertion by monitoring the heart rate of the patient and providing an indicator if and when the patient is about to move out of the physical exertion range prescribed by the physician/clinician/trainer. The systems and methods allow a patient to function with confidence and insight, while avoiding the danger of over-exertion, both cognitively and physically to the point of recovery regression.

The systems and methods comprise a number of components, including an EEG headset; a heart-rate monitor; a backend data processing system which uses machine learning to process data and detect neuromarkers of cognitive and physical overexertion; and a mobile application that patients are able to use to guide their recovery process in real time. The mobile application comprises a number of functions. First, it allows patients to track their activities and symptoms and register symptom triggers (i.e. bright light). Next it pairs with the EEG headset to alert the patient when they are showing signs of cognitive (through EEG) and physical (through heartrate) over-exertion. Finally, patients are able to see their activity history and share this data with their physician through a web-based portal. In some embodiments, the systems and methods include a web-based portal that allows authorized doctors to track recovery of their patients. The physician/clinician/trainer can see the data collected over a selected range of time to effectively manage symptom triggers and to continuously keep the patient moving forward in their recovery process.

The systems and methods disclosed herein shorten concussion recovery, which is measured by the time it takes to return to play, work or school, in addition, the systems and methods disclosed herein improve mental health and self-efficacy, as measured through anxiety, depression and self-efficacy scores.

Concussion recovery needs a patient to maintain a balance between pushing oneself physically and mentally, that is being careful neither to be inactive, nor overdo activities. Optimal rehabilitation comes from managed recovery, which is provided by the methods and systems disclosed herein.

The systems and methods disclosed herein may or may not work when there is: evidence of structural damage to the brain as shown, for example, by imaging; a history of substance dependence by the concussed patient; and/or a history of epilepsy.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

To easily identify the discussion of any particular element or act, the most significant digit or digits in a reference number refer to the figure number in which that element is first introduced. Like reference numbers and designations in the various drawings indicate like elements.

FIG. 1 illustrates an example in accordance with some embodiments.

FIG. 2 is an apparatus for generating a brain break alert, in accordance with some embodiments.

FIG. 3 illustrates a block diagram in accordance with some embodiments.

FIG. 4 illustrates a master flowchart in accordance with some embodiments.

FIG. 5 illustrates an example of how Total Brain Energy changes in accordance with some embodiments.

FIG. 6 illustrates a flowchart in accordance with some embodiments.

FIG. 7 illustrates data processing architecture in accordance with some embodiments.

FIG. 8 illustrates a regression decision tree in accordance with some embodiments.

FIG. 9 is a flow diagram of a method to train a machine learning model, in accordance with some embodiments.

FIG. 10 is a flow diagram of a method for generating a brain break alert, in accordance with some embodiments.

FIG. 11A illustrates a front view of an EEG headset worn by a patient, in accordance with some embodiments.

FIG. 11B illustrates a rear view of the EEG headset, in accordance with some embodiments.

FIG. 12A illustrates a rear perspective view of an EEG headset in accordance with some embodiments.

FIG. 12B illustrates the EEG headset of FIG. 12A in accordance with one embodiment.

FIG. 12C illustrates the EEG headset of FIG. 12A in accordance with some embodiments.

FIG. 12D illustrates the EEG headset of FIG. 12A in accordance with some embodiments.

FIG. 13 illustrates several views of an EEG headband in accordance with some embodiments.

FIG. 14A illustrates a front view of an EEG headband in accordance with some embodiments.

FIG. 14B illustrates a side view of an EEG headband in accordance with some embodiments.

FIG. 14C illustrates cut-away side view of a board casing in accordance with some embodiments.

FIG. 15 illustrates a perspective view of an EEG headband in accordance with some embodiments.

FIG. 16 illustrates an electrode montage in accordance with some embodiments.

FIG. 17 illustrates partial assembly of an EEG headband in accordance with some embodiments.

FIG. 18 illustrates an in-ear electrode in accordance with some embodiments.

FIG. 19 illustrates EEG headset in accordance with some embodiments.

### DETAILED DESCRIPTION

Embodiments of the present invention relate to method and apparatus for concussion recovery, including generating a brain break alert for a patient suffering from a concussion. The patient wears an electroencephalographic (EEG) headset around their head, and engages in a cognitive activity or task. The EEG headset spans the forehead, temples and ears of the patient and includes electrodes for capturing EEG data of the patient through direct contact of the electrodes with the head of the patient. The EEG headset is communicably coupled with a user device, such as a handheld device accessible by the patient or a caregiver to the patient, for example, a smart phone or a tablet, among several others, and the EEG headset sends the captured EEG data to the user device. The user device is communicably coupled to a system server via a network, such as, for example, the Internet, and the user device sends the EEG data to the system server. The system server, based on the EEG data, determines the brain energy used (BEU), and whether the patient should take a break to aid or optimize concussion recovery. In some embodiments, the system server determines an instantaneous mental workload (IMWL) based on the EEG data, and the system server uses the IMWL to determine a brain energy used (BEU) by the patient while performing the cognitive activity. The system server utilizes the brain energy used (BEU) to determine a brain energy available (BEA) to the patient. When the brain energy available (BEA) falls below a brain break alert threshold, the system server instructs the user device to generate a brain break alert to the patient, indicating that it is recommended that the patient take a break in order to aid recovery.

In some embodiments, the system server utilizes a machine learning (ML) model to determine IMWL based on the EEG data. The ML model is custom-trained according to the patient, using training data from the patient. The training data includes the EEG data of the patient engaged in a training phase activity, and the patient's self-assessment of the mental workload at the conclusion of the training phase activity. The training phase activities includes one or more of rest, eyes open, eyes close, visuospatial tasks, arithmetic, logic puzzles, executive function, memorizing, or varying level of difficulty thereof.

The EEG headset is designed to increase comfort for the patient when worn on the head of the patient, and to collect the EEG data more reliably and accurately than has been previously possible.

Wearable technology is combined with proprietary machine learning algorithms customized according to a patient, enables a personalized medicine approach to brain health and brain injury recovery. Patients use the ubiquitous smartphones via a mobile application thereon to track their own activity, symptoms, and triggers, which enables patients to engage with cognitive and physical activity knowing they will receive an alert from their Neurovine application before they overexert physically and cognitively, that could hinder their recovery and/or progress. In some embodiments, the disclosed apparatus including machine learning algorithms are capable of generating an alert 0.5 seconds or less, from the time the EEG data is measured at the patients' scalp. Moreover, the algorithms disclosed herein grow with the patient, allowing the patient to increase their activity as the patient heals.

Machine-learning-based systems and methods for concussion recovery that comprise a software platform that uses EEG from a portable headset to create a personalized concussion rehabilitation tool. The systems and methods provide personalized, real-time data on a patient's cognitive load and deliver an indicator when the patient is progressing towards a fatigued brain state, prompting the patient to take a “brain break”. The systems and methods do the same for physical exertion by monitoring the heart rate of the patient and providing an indicator if and when the patient is about to move out of the physical exertion range prescribed by the physician/clinician/trainer. The systems and methods allow a patient to function with confidence and insight, while avoiding the danger of over-exertion, both cognitively and physically to the point of recovery regression.

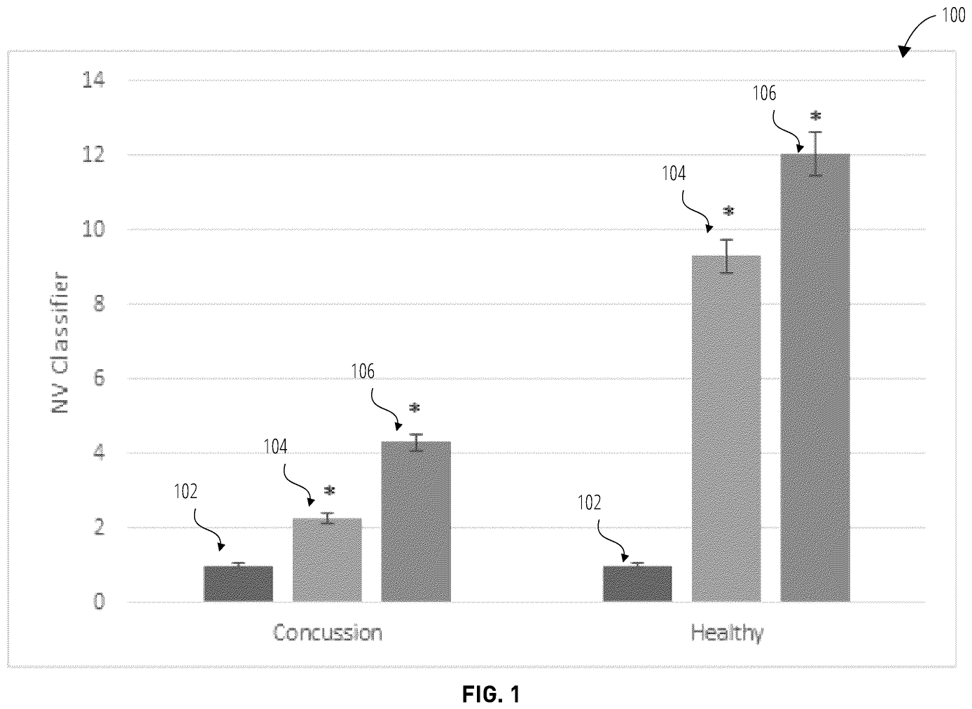

As an illustration of the difference between cognitive over exertion, FIG. 1 shows bar graphs from a study of one of the many EEG features that are used to measure mental workload at rest 102; during healthy engagement 104 and during overexertion 106 in concussion patients and healthy individuals (control subjects). The feature shown in FIG. 1 is alpha frontal/theta frontal. In the study, 15 concussion patients and 15 healthy individuals were tested, and the EEG feature demonstrated that rest 102 scenarios were similar for both the concussion group and the healthy group, however, there was a significant difference in healthy engagement 104 scenarios (p<0.05) and overexertion 106 scenarios (p<0.05) of the concussion group versus the healthy group.

While both healthy individuals and concussion patients show a similar trend, however, the concussion patients reach the state of cognitive overexertion quickly (average of 30 minutes) compared to the healthy individuals to show signs of cognitive overexertion (about 90 minutes). The data indicates that concussion patients have a dampened evolution compared to healthy individuals. It is theorized that this may be caused partly by the inability of the concussion patients to synchronize cognitive effort in an efficient way post-concussion, and partly by higher resting or baseline data found in concussion patients.

The embodiments disclosed herein allow for building computational model(s) that are specific to a patient's own brain data (EEG data), that is, personalized to the patient. Further, the computational methods disclosed herein enable the patient to see real-time analytics on the state of their brain energy, and know when it's time to take a “brain break” before they over-exert themselves, that may delay concussion recovery. The analytics can be accessed by the patient conveniently on the user device accessible to the patient, for example, smartphones, tablets, computers and the like. The embodiments disclosed herein also allow for a patient to share data (for example, through a clinical portal) with healthcare professionals, providing data-driven insights to the healthcare professionals, that may help the healthcare professionals develop a customized recovery plan for the patient. Some embodiments include machine learning to build the computational model(s) employing machine learning based on the EEG data of the patient, for example, captured using an EEG headset worn by the patient.

Some embodiments disclosed herein include a portable, wireless EEG headset, a heart-rate monitor, a mobile application, privacy-legislation (e.g. HIPPA) compliant backend storage mechanism, and a machine-learning analytics engine. In some embodiments, the embodiments also include a clinical web portal.

The embodiments disclosed herein provide real-time data and/or analytics for patients and clinicians during the patients' concussion recovery process, during which the patients are instructed to limit their cognitive and/or physical activity or task. According to some embodiments, for physical activity, a patient places the heart rate monitor on the chest or wrist, opens the mobile application on a user device, such as a smartphone or a tablet, and connects the heart rate monitor to the user device via a wired connection or a wireless connection, such as via Bluetooth. Next, the patient selects ‘physical activity’ in the application, presses start, and performs a physical exercise, for example, running, walking or non-contact drills that may be approved by a supervising clinician. The mobile application alerts the patient when the patient is exercising too hard and needs to slow down and/or take a break, so as to prevent aggravating the concussion.

For cognitive activity, the patient places the EEG headset on the head, opens the mobile application on the user device and connects the headset to the device via a wired connection or a wireless connection, such as via Bluetooth. Next the patient selects cognitive activity in the application, presses start, and performs a cognitive task, for example, reading, writing, typing, mathematical problems, puzzles, and the like. The mobile application provides an alert when the patient begins to approach cognitive overexertion as determined using the embodiments described herein.

All of the data collected during these activities is tracked and compiled into trends that can be displayed in an intuitive format for the patient and a more detailed format in the web portal for the clinician, physician or trainer. The techniques disclosed herein allow concussion patients to perform cognitive and physical activity post-concussion with confidence, knowing that they will receive an alert if they are over exerting physically or mentally in a manner that is likely to hinder their recovery from concussion. In some embodiments, a patient's data history is tracked and presented to the patient or the healthcare providers to see the patient's progress, identify symptom triggers, if any, make informed medical decisions for their patients.

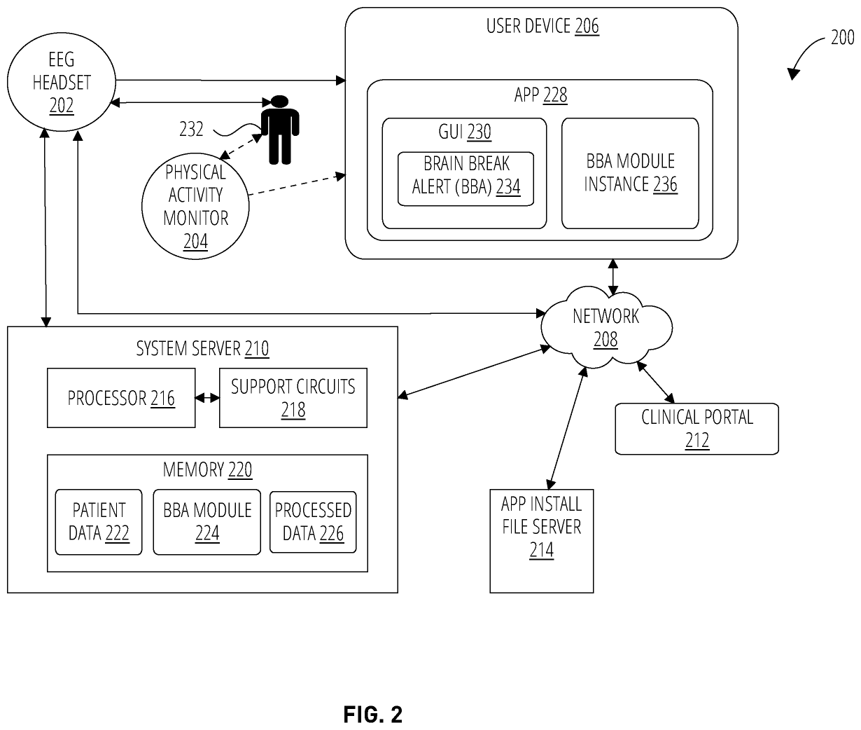

FIG. 2 is an apparatus 200 for generating a brain break alert, in accordance with some embodiments. The apparatus 200 includes an electroencephalographic EEG headset 202 communicably coupled to a user device 206. The EEG headset is configured to collect EEG data of a patient, for example, the patient 232. In some embodiments, a physical activity monitor 204 is included and communicably coupled to the the user device 206, and the physical activity monitor 204 is configured to collect physiological data of the patient 232. The apparatus 200 further includes a system server 210, a clinical portal 212, and an app install file server 214. The user device 206, the system server 210, the clinical portal 212 and the app install file server 214 are communicably coupled to each other via the network 208.

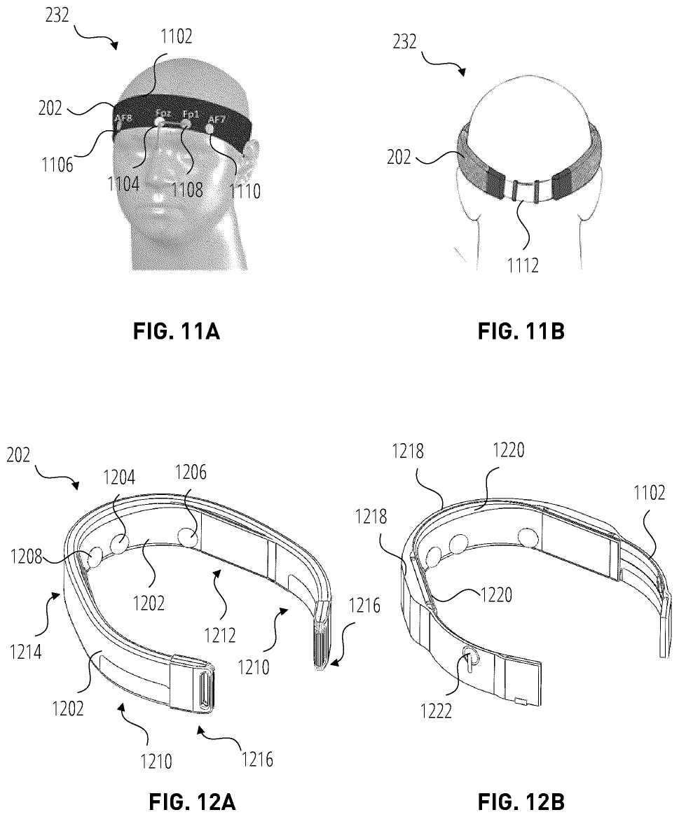

The EEG headset 202 is configured to be worn by the patient 232 over the head of the patient 232, and is configured to collect and send EEG data of the patient 232 to the user device 206. The EEG data includes data from one or more contact points of sensors or electrodes of the EEG headset with the head, such as the forehead, the temple area, ear or over the ear, back of the head, top of the head, face, neck and the like, or any other areas from where EEG data may be captured by the electrodes. In some embodiments, the EEG headset 202 is configured to send the EEG data directly to the system server 210 via the network 208 and/or via the the user device 206, or via a direct link, such as a wired or a wireless connection as known in the art, and the EEG headset 202 may include devices to communicate a brain break alert (BBA) to the patient 232. Such devices may include a vibrating device (such as those found in smart phones), configured to vibrate the EEG headset 202, a speaker configured to generate a sound from the EEG headset 202, a display screen physically and/or communicably coupled to the EEG headset 202, among other suitable devices well known in the art, to alert the patient 232 wearing the EEG headset 202. The EEG headset 202 is discussed in greater detail with respect to FIG. 11A-FIG. 19.

The physical activity monitor 204 is a device worn by the user and capable of measuring physical activity of the patient 232, for example, heart rate or other physical parameters, such as breaths per minute, using techniques as known in the art.

The user device 206 is a computer, such as a personal computer (PC), a laptop, a tablet, a smartphone, and the like, used, for example, by the patient 232 or a caregiver of the patient 232. The user device 206 comprises a CPU, support circuits and a memory (not shown). The CPU may be any commercially available processor, microprocessor, microcontroller, and the like. The support circuits comprise well-known circuits that provide functionality to the CPU such as a user interface, clock circuits, network communications, cache, power supplies, I/O circuits, and the like. The I/O circuits include a display, for example, various standard or touch-based displays, such as computer monitors as generally known in the art. In some embodiments, the user interface comprises a keypad, electronic buttons, speaker, touchscreen, display, or other user interaction mechanism. The memory is any form of digital storage used for storing data and executable software. Such memory includes, but is not limited to, random access memory, read only memory, disk storage, optical storage, and the like. The memory stores computer readable instructions corresponding to an operating system (not shown), an app 228 and a graphical user interface GUI 230 of the app 228, which is displayed on the display.

The GUI 230 is capable of presenting a brain break alert (BBA) 234 on the user device 206, for example, to attract the attention of the patient 232. The brain break alert (BBA) 234 may include an auditory, visual, vibratory or any other output that can be generated by the user device 206 and perceived by a human, for example, the patient 232. The app and the GUI 230 are capable of several other functions in addition to communicating the brain break alert (BBA) to the patient 232, such as presenting processed data and analytics to the patient 232, recommended activities to be performed by the patient 232, a recovery and/or training program including a series of activities for the patient 232, receiving patient inputs with respect to the activities of the patient 232, for example, for training the ML model according to engagement protocol(s) administered by a clinician to the patient 232 or automated administration of the engagement protocol(s) on self-service basis by the patient 232, receiving from the patient, a self-report of their cognitive exertion associated with the cognitive activity, among others.

The app 228 can, via the GUI 230, also guide the patient to perform an impedance check to ensure good electrical contact of the electrodes with the patient's skin and guiding them to use saline solution or other conductive gels or other aids that improve the conductivity (and therefore lower impedance) with the electrodes, and guide the patient to adjust the tension and positioning of the EEG headset 202, guide the patient to connect the EEG headset 202 via wireless means (e.g. Bluetooth or WiFi), present a connectivity alert to the patient if the smartphone is not receiving or losing data packets, or receiving corrupted packets of EEG data from the EEG headset, provide a noise alert to the patient indicating there are sources of biological noise, environmental noise or improper contact with the patient's skin (e.g. headband has moved) that are corrupting or interfering of the EEG signal such that the EEG headset 202 does not receive signals from the patient's brain, and instructs the patient on actions the patient can take to remedy the issues, such as reducing sources of unwanted noise (e.g., stop chewing gum). Further, the app 228 can also guide the patient to set the Brain Break Alert Difficulty Levels, alert the patient of a battery level of EEG headset, and if the EEG headset is connected, among several other common features associated with fitness wearables.

Each sample of EEG also has a timestamp or index associated with it that is added by the headband and transmitted to a mobile app on the user device. The timestamp and or index can be used by the mobile app to determine if any EEG samples were lost or corrupted when transmitted from the headband to the mobile device. The EEG headset can also send impedance values per electrode either concurrently with EEG samples or as a separate measurement when EEG measurements have stopped. The EEG headset can also send battery fuel gauge information to the mobile app to let the user know how much battery life is left with this charge. The mobile app can also send commands to the EEG headset to, for example, start impedance measurements or run tests of the EEG headset or normal operation.

The network 208 comprises the Internet, or a wide area network (WAN) or a combination thereof, and may include one or more such networks, spanning the various devices as illustrated in FIG. 2. All the components of the apparatus 200 are connected to the network 208 or to each other as illustrated in FIG. 2, using known methods and components.

The system server 210 may be a general-purpose computer or other electronic processing device that is programmed to perform functions related to embodiments of the present invention. The system server 210 comprises a processor 216, support circuits 218, and a memory 220 containing instructions and algorithms. The processor 216 may be any commercially available processor, microprocessor, microcontroller, and the like. The support circuits 218 comprise well-known circuits that provide functionality to the CPU such as a user interface, clock circuits, network communications, cache, power supplies, I/O circuits, and the like. Alternative embodiments may use control algorithms on a custom Application Specific Integrated Circuit (ASIC) to provide the functionality provided by the any combination of the processor 216, the support circuits 218 and the memory 220. In some embodiments, the user interface comprises a keypad, electronic buttons, speaker, touchscreen, display, or other user interaction mechanism.

The memory 220 may be any form of digital storage used for storing data and executable software. Such memory includes, but is not limited to, random access memory, read only memory, disk storage, optical storage, and the like. The memory 220 stores computer readable instructions corresponding to an operating system (not shown), patient data 222 received from the EEG headset 202 and/or heart rate data received from the physical activity monitor 204, a brain break alert BBA module 224 configured to process the patient data 222 to generate processed data 226, which includes, among others, an instruction to generate the brain break alert, for example, on the user device.

The patient data 222 is data as captured by the EEG headset 202 or the physical activity monitor 204 and sent to the system server 210, for example, via the user device 206, the network 208 or directly from the EEG headset 202. The patient data 222 includes, for example, the EEG data including electric signals from the portions of the head of the patient as captured by the electrodes of the EEG headset 202, or the physical activity data including electric signals of the patient 232. In some embodiments, the patient data 222 further includes the sex and/or gender, age of the patient, other aspects of the patient's medical record that can inform the concussion treatment plan, for example, the time, date, severity, of each symptom experienced by the patient, the type of activity performed by the patient in the past, associated start and end times, and total BEU per sessions, the BEI for the patient, the BBAT associated with each BBA difficulty level, the body system(s) of the patient that are the root cause for the symptoms they are experiencing, such as cervical spine, oculomotor, brain metabolism, physical or vestibular.

The BBA module 224 is a software module which processes the patient data 222 received from the patient 232, and among other things, generates instructions to communicate a brain break alert (BBA) 234 to the patient 232, and generates, manages and tracks the treatment plan of the patient 232.

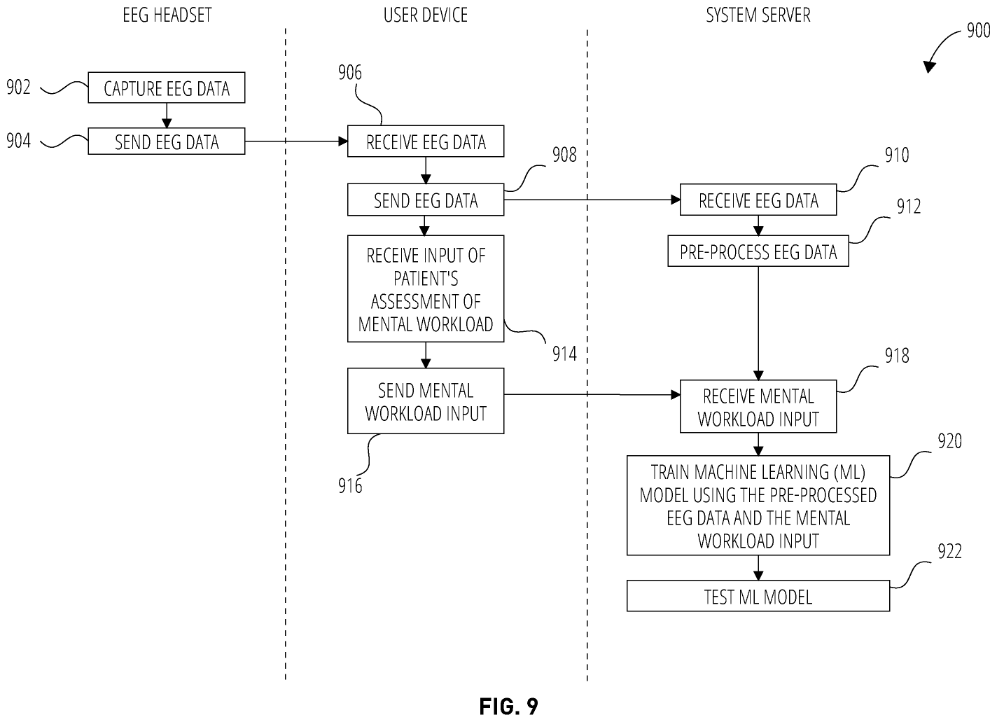

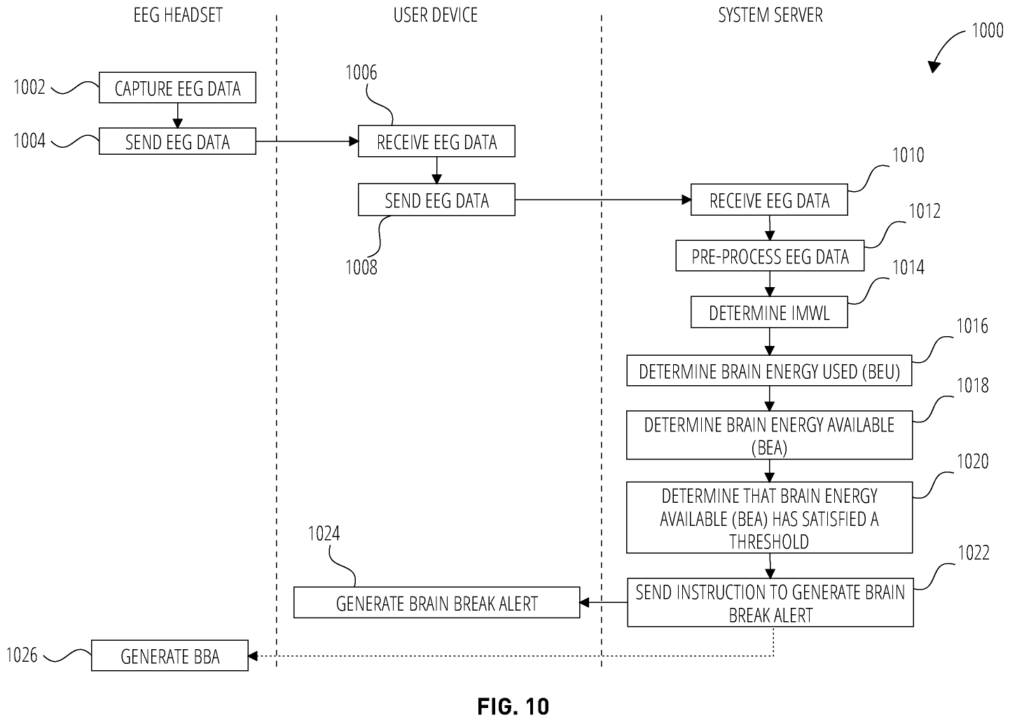

The BBA module 224 is configured to determine, based on the EEG data comprised in the patient data 222, brain energy used (BEU) by the patient 232 during or for a given task (cognitive activity). In some embodiments, the BEU is a cumulation of instantaneous mental workload (IMWL) determined by the BBA module 224 based on the EEG data. A cumulation of IMWL includes a cumulation of a function of the IMWL, such as normalized IMWL, a sigmoid function of the IMWL, among others. In some embodiments, the BBA module 224 comprises a fixed set of instructions to determine IMWL based on the EEG data. In some embodiments, the BBA module 224 comprises a machine learning (ML) model to determine the IMWL based on the EEG data, for example, as discussed below with respect to FIG. 9 and FIG. 10 below.

Based on the BEU, and a configurable and/or predefined initial brain energy (BEI) of the patient 232, the BBA module 224 is configured to determine the brain energy available (BEA) to the patient 232. The BEA of the patient 232 is tracked over time and compared to a brain break alert threshold (BBAT). The BBAT is the brain energy level of the patient 232, below which, it is assumed that exerting further mentally would deplete the brain energy further, and would not assist in the recovery of the patient 232, may harm the recovery process of the patient 232. In some embodiments, the BBAT is determined based on the patient's self-evaluation of brain energy while performing certain tasks. The BBAT has the same unit as the BEA, and when the BEA becomes equal to or lower than the BBAT, the BBAT threshold is deemed to have been satisfied. According to the embodiments disclosed herein, satisfying the BBAT implies that the BEA to the patient 232 is lower than what is optimal for recovery of the patient 232.

Accordingly, upon determining that the BBAT has been satisfied, the BBA module 224 sends an instruction to generate a brain break alert (BBA) 234. In some embodiments, the BBA module 224 send the instructions to the user device 206, which generates the brain break alert (BBA) 234 on the GUI 230. In some embodiments, the BBA module 224 sends the instructions to the EEG headset 202, directly, via the network 208 and/or via the user device 206, which may include devices to communicate a brain break alert (BBA) to the patient 232.

In some embodiments, the BBA module 224 is configured to create a treatment plan based on the EEG data, and/or optionally inputs from a clinician, for example, received via the clinical portal 212. In some embodiments, the BBA module 224 modifies the treatment plan based on clinician input, patient's progress as determined by processing the patient data 222. In some embodiments, the BBA module 224 processes the patient data 222 to generate analytics, including analytics based on the IMWL, BEU, BEA among others.

The processed data 226 includes patient data 222 processed by the BBA module 224, where the processing includes pre-processing the data including cleaning the data to remove noise, processing the data to predict or determine instantaneous mental workload (IMWL) for the patient 232, to determine the brain energy used (BEU) by the patient 232, to determine the brain energy available (BEA) to the patient 232 as a function of a predefined initial brain energy (BEI) of the patient 232, to determine when a brain break alert threshold has been satisfied by the BEA, and the brain break alert (BBA) 234 should be generated for the patient 232, among other processing. In some embodiments, the processed data 226 includes trends of the patient 232's parameters (IMWL, BEU, BEA) across activities and across days of treatment, and a treatment plan for the patient 232 including cognitive activities for the patient 232.

The BBA module 224 presents one or more of the processed data 226 to the patient, for example, via the user device 206 on the GUI 230, and in some embodiments, the BBA module 224 may also send part or complete processed data 226 to the clinical portal 212 for review by a clinician. When the brain break alert threshold is satisfied, the BBA module 224 sends the instructions to generate the brain break alert (BBA) 234, for example, to the user device 206, and in some embodiments, directly to the EEG headset 202.

In some embodiments, after the BBA module 224 is trained, an instance of the BBA module may be implemented on the user device 206, for example, as BBA module instance 236. The BBA module instance 236 is capable of performing all functions of the BBA module 224 from the user device 206. In some embodiments, the BBA module instance 236 is a part of the app 228, and in some embodiments, the BBA module instance 236 interacts with the app 228. In some embodiments, the BBA module 224 is configured to send an installation file for the BBA module instance 236 to the user device 206.

The clinical portal 212 is a computer, similar to the user device 206, and can display all processed data 226, patient data 222 to a clinician/healthcare provider of the patient 232. The clinician may, via the clinical portal 212, administer or modify treatment plan of cognitive activities or tasks of the patient 232, which are accessible to the patient 232 via the GUI 230 of the user device 206, using techniques as known in the art. In some embodiments, the clinician may, via the clinical portal 212, create and/or modify the treatment plan included in the processed data 226 on the system server 210. The clinician can also adjust, via the clinical portal 212, the BBA difficulty level based on the symptoms reported by the patient, declare that the patient is able to return to work or play because they are experiencing few symptoms, and/or their cognitive and physical capacity has returned back to normal levels before the concussion occurred.

The app install file server 214 is a computer, similar to the system server, and includes an installation file for the app 228. The installation file for the app 228 can be downloaded at the user device 206, for example, by the patient 232 by accessing the app install file server 214, and executed on the user device 206 to install the app 228 on the user device 206. As such, the installation file for the app 228 includes computer-executable instructions corresponding to the app 228 and for installing the app 228 on the user device 206.

The apparatus 200, and various components therein, are capable of performing the methods and all steps therein described herein in “real time,” which will be understood to mean as soon as possible given the physical constraints of the apparatus and components thereof, for example, processing times, communication times and the like. Therefore, according to some embodiments, the brain break alert (BBA) 234 is generated in real time, that is, while the patient 232 is engaged in an activity. In some embodiments, delays may be introduced at one or more steps of the methods disclosed herein deliberately, and all such variations are included in real time. In some embodiments, the methods may be performed passively, that is, after the patient 232 has performed one or more activities, but during such activities the EEG data of the patient 232 has been captured. Such passive methods may be used to determined if the patient 232 crossed the BBAT, that is, continued mental exertion for a longer duration than is considered optimal.

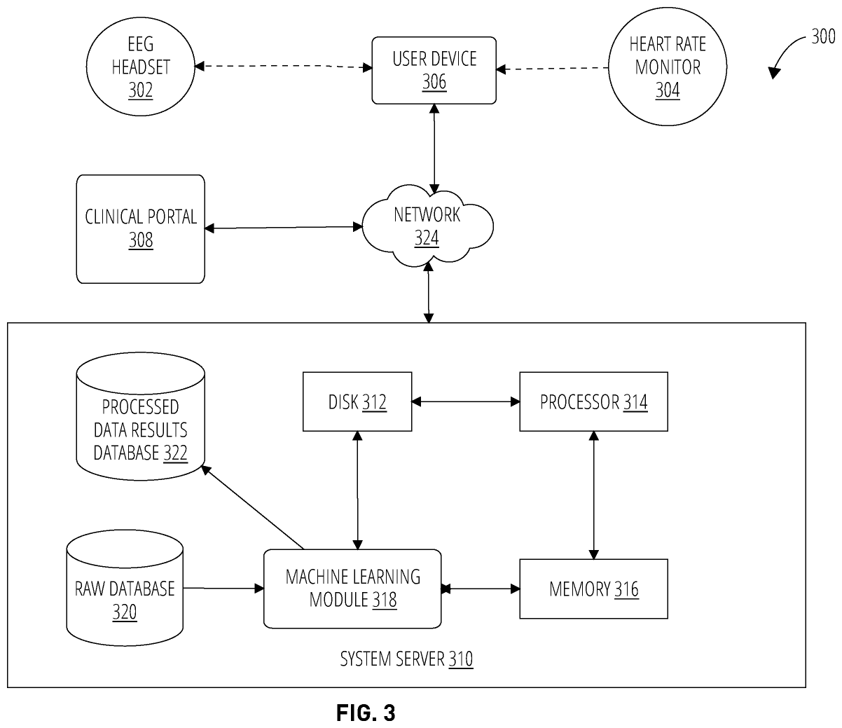

FIG. 3 illustrates an apparatus 300 in accordance with some embodiments.

In FIG. 3, data from the EEG headset 302, for example, similar to the EEG headset 202 and/or heart rate monitor 304, for example, similar to the physical activity monitor 204 is transferred via BlueTooth to a user device 306, for example, similar to the user device 206. The data from the EEG headset 302 and/or the heart rate monitor 304 is then sent, via an API call, to the system server 310 for analysis. Real-time patient data (EEG, heart rate) is securely transferred from the user device 306, analyzed and stored in system server 310 which is compliant with local privacy legislation. For example, in the United States, system server 310 is compliant with the Health Insurance and Portability and Accountability Act (HIPAA).

Using a customized mobile app on user device 306, a patient has the ability to connect an EEG headset 302 or Heart rate monitor 304 that streams live-data to the user device 306. This data is streamed from the EEG headset 302 or heart rate monitor 304 to the user device 306 using Bluetooth Low Energy (BLE), or any other local low energy transmission platform. Next, upon the patient's approval of data transfer, analysis and storage guidelines, the collected data is communicated to system server 310, using the Hypertext Transfer Protocol-Secure (HTTPS). In some embodiments, the system server 310 is housed in the country where the technology is being used.

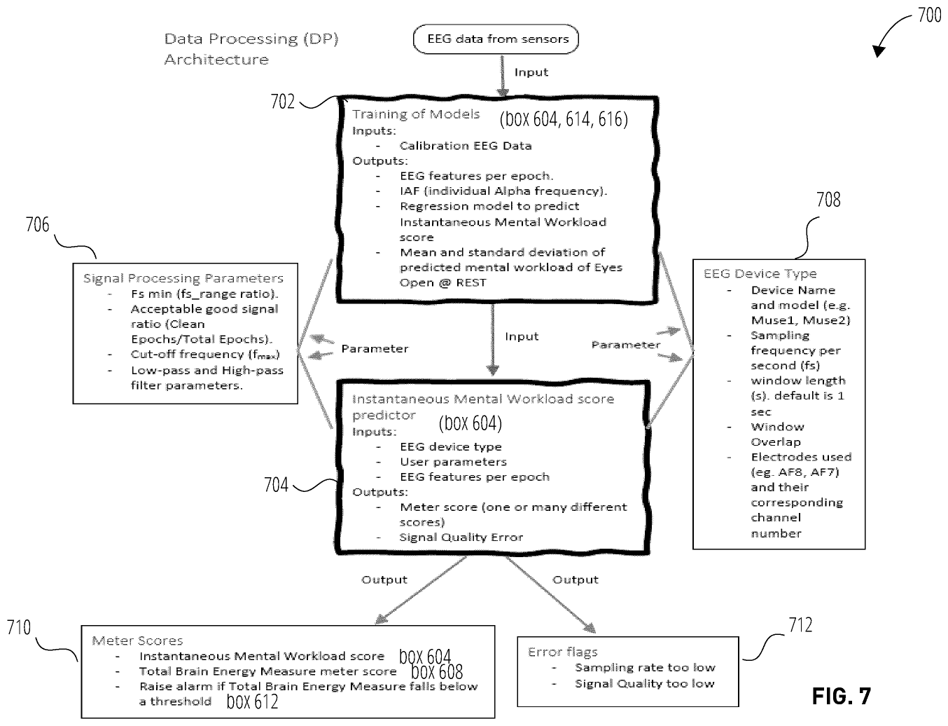

When data arrives into raw database 320, it is analyzed using the machine learning module 318 in quasi real-time using the data processing architecture outlined in FIG. 7. After the data is analyzed, it is transferred and stored in the processed data results database 322 and then communicated back to the user device 306, thereby providing the patient with useful and powerful insights regarding their cognitive and physical health.

Separately, clinicians (i.e. physicians or care givers) can have access to patient data through the clinical portal 308. In some embodiments, clinicians have access if a patient specifies that a specific clinician can access their data. The clinical portal 308 uses the same backend used by the mobile app on user device 306, hence all the security measures outlined above are automatically inherited by clinical portal 308.

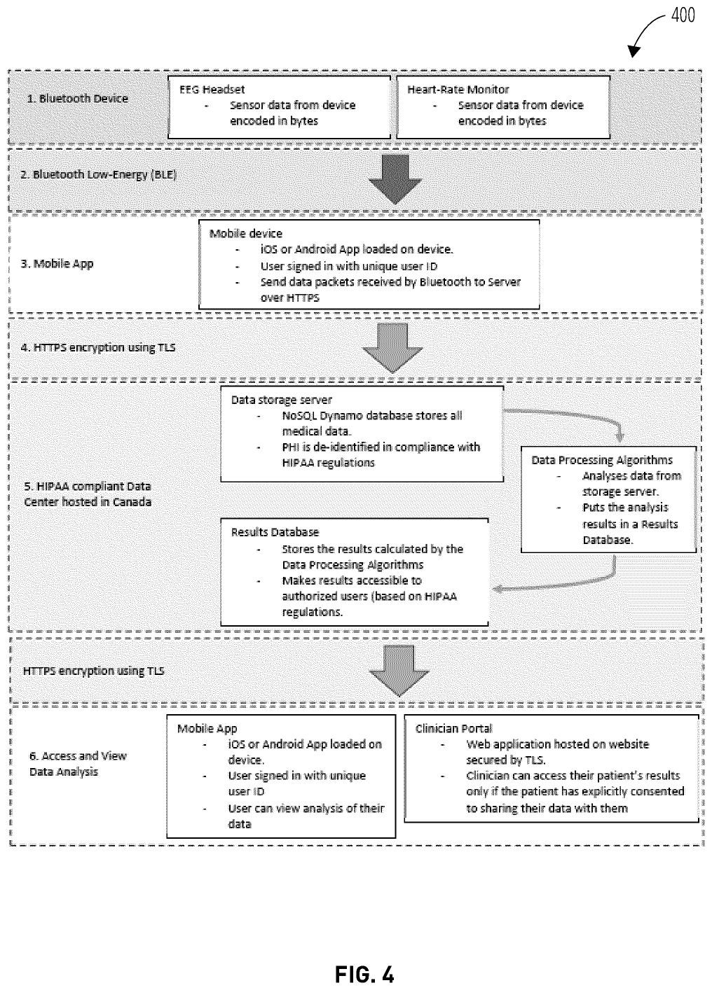

FIG. 4 illustrates a flowchart 400, in accordance with some embodiments. The flowchart 400 comprises the following components:

Bluetooth Device

These are the medical sensors used to obtain patient biometric data. These devices are all Bluetooth Low Energy (BLE) compatible; they contain BLE radios to send and receive packets of data to a BLE compatible device.

Bluetooth Low Energy (BLE)

This is the data transmission protocol used to send biometric sensor data to a BLE enabled mobile device running a proprietary application. The data is secured using AES-CMAC encryption (aka AES-128 via RFC 4493, which is FIPS-compliant) during communications when the devices are unpaired. This allows the data to flow from the Bluetooth device to the mobile app. Each sample of EEG also has a timestamp or index associated with it that is added by the headband and transmitted to a mobile app on the user device. The timestamp and or index can be used by the mobile app to determine if any EEG samples were lost or corrupted when transmitted from the headband to the mobile device. The EEG headset can also send impedance values per electrode either concurrently with EEG samples or as a separate measurement when EEG measurements have stopped. The EEG headset can also send battery fuel gauge information to the mobile app to let the user know how much battery life is left with this charge.

Mobile App

This represents proprietary software running on an Android or iOS mobile device. The app receives data from the Bluetooth device, processes the data to obtain relevant biometric signals, and sends the data to the Data Center to be processed. The data is sent from the mobile device to the data center over the internet using HTTPS encryption with TLS. The mobile app can also send commands to the EEG headset to, for example, start impedance measurements or run tests of the EEG headset or normal operation.

HTTPS Encryption Using TLS

This is the data transmission protocol used to send biometric sensor data from the mobile device to the data center over the internet. The data transfer is performed using Hypertext Transfer Protocol-Secure (HTTPS).

HTTPS ensures that any communication is authenticated and encrypted using the Secure Sockets Layer protocol (SSL); which utilizes the asymmetric public key infrastructure (i.e. private key-public key exchange).

HIPAA Compliant Data Center Hosted in Canada

This component stores the data, processes the data to extract meaningful information, and stores the results of the data processing. Data in the databases is fully encrypted using hardware security modules validated under FIPS 140-2. Data is also redundantly stored across multiple database servers, that follow the same encryption standards, at different physical locations. Finally, data transferred between such locations are governed by a Virtual Private Cloud that may allow connectivity to the database, and in some embodiments, the Virtual Private Cloud may allow connectivity exclusively to the database.

Access and View Data Analysis

This component allows access to the results for authorized patients, for example, as authorized according to HIPAA regulations. This includes the patient who wants to see their data, as well as a caregiver/physician that the patient has consented to view their data.

Access to the data is implemented using HTTPS encryption described previously, and it is available on the patient's mobile App as well as the caregiver/physician's Clinical Portal.

The Clinical Portal is a web application that allows authorized patients to securely log in and view their patient's data over the internet.

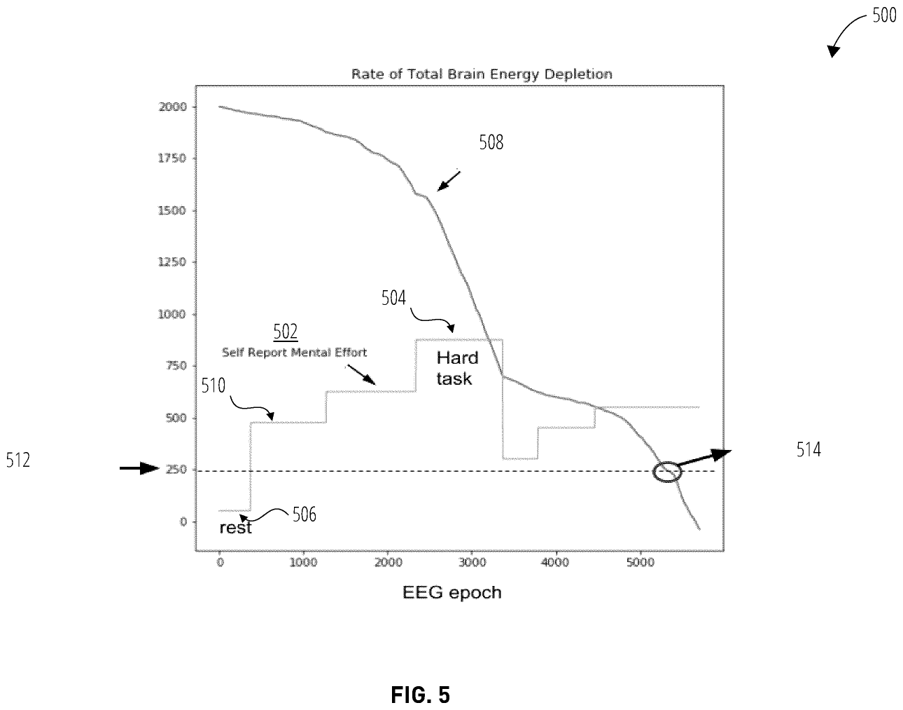

FIG. 5 illustrates a graph 500 of how Total Brain Energy/Brain Energy Available (BEA) 508 changes for a patient, for example, the patient 232 while performing certain activities or tasks. The x-axis is the EEG epoch and the left-hand y-axis is the brain energy available (BEA) in brain energy units. The right hand y-axis is the level of self-report mental effort of a patient.

Line 502 indicates the level of self-report mental effort of a patient based on the mental effort tasks as described above. Each level represents a different task. The highest level 504 of line 502 corresponds to the “Hard Task” (i.e. a very difficult mental exercise for the brain), while the lowest level 506 corresponds to “rest” (i.e. little mental effort for the brain). FIG. 5 illustrates a strong correlation of how quickly Total Brain Energy/Brain Energy Available (BEA) 508 depletes the more difficult the task. During the Hard Task (highest level 504 of line 502), the Total Brain Energy/Brain Energy Available (BEA) 508 drops rapidly from about 1600 to about 675—whereas during easier tasks, the Total Brain Energy/Brain Energy Available (BEA) 508 drops slowly. For example, during the relatively easy task 510, the Total Brain Total Brain Energy/Brain Energy Available (BEA) 508 drops from about 2000 to about 1900.

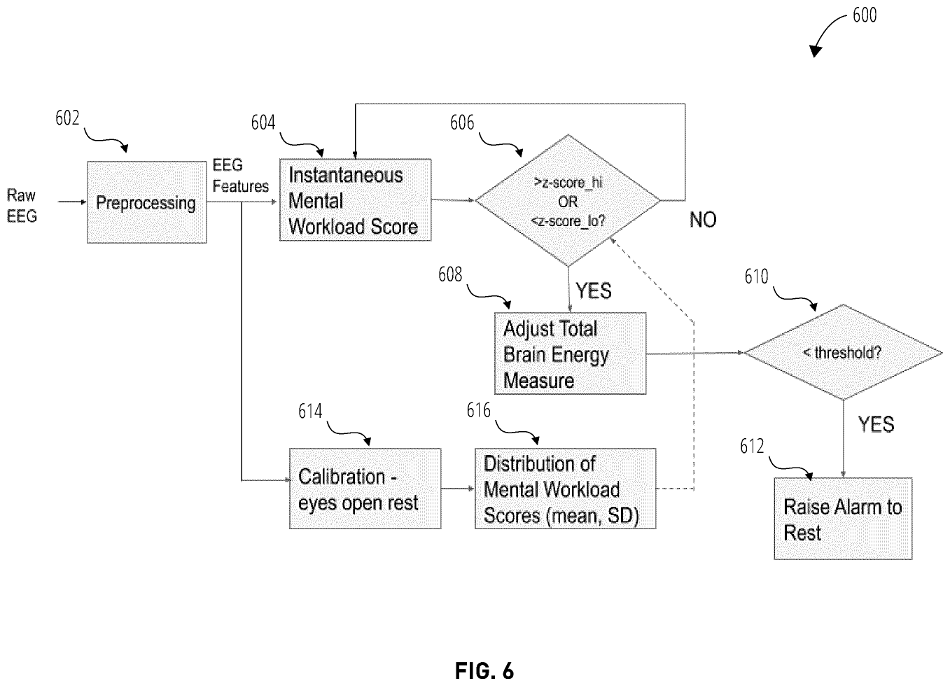

The graph 500 shows that after almost 6000 epochs (almost one hour at working hard across 6 different levels of mental tasks), the patient's Total Brain Energy/Brain Energy Available (BEA) 508 has fallen below the BBAT 512 of 250 (see box 610 of FIG. 6). When Total Brain Energy/Brain Energy Available (BEA) 508 falls below BBAT 512 for this patient, then the Alarm to Rest 514 is raised, for example, see box 612 of FIG. 6.

FIG. 6 illustrates a flowchart 600, in accordance with some embodiments.

Box 602

At box 602, raw voltage values of EEG from one or more electrodes from a person's scalp are converted into digital samples. The digitized EEG signals are processed, and cleaned, by filtering the EEG data and removing biological noise and movement artifacts, before being subject to machine learning.

Box 604

At box 604, the EEG signal is divided into overlapping epochs such as one second of digitized raw voltage values. Features of each epoch are calculated. The features of the EEG signal can be frequency domain, time domain or spatial. The features can also be linear or non-linear. The features are fed to box 604, which calculates an Instantaneous Mental Workload (IMWL) Score. The IMWL Score can be based on a Machine Learning method such as Linear Regression, Random Forest Regression or a non-linear model.

Box 606 and Box 608

Box 606 is a decision that compares the IMWL score against two thresholds, z-score_lo and z-score_hi. In one embodiment, the z-score is used to compare with the IMWL score. The z-score is a statistic that measures the distance in standard deviations of a sample against a reference distribution. In some embodiments, the reference distribution is the IMWL score of a five-minute recording of eyes open rest of the patient calculated by box 616. Five minutes is the suggested length of time; however, this may vary from patient to patient and the length of time required for calibration may be lower with future improvements in pre-processing and machine learning techniques. In one embodiment, one standard deviation above the mean of the reference distribution of IMWL establishes the threshold for z-score_hi (i.e. z-score_hi=+1) and one standard deviation below the mean establishes the threshold for z-score_lo (i.e. z-score_hi=−1). If the IMWL score is greater than the threshold z-score_hi, then the patient's mental workload is high enough to deplete the patient's Total Brain Energy (or the brain energy available (BEA) to the patient 232). When this happens, the Total Brain Energy is adjusted downwards in proportion to the level of IMWL above z-score_hi in box 608. See FIG. 5 for an example of how Total Brain Energy depletion is correlated with mental effort (IMWL). Also, if the IMWL score is less than the z-score_lo threshold to indicate rest, then the Total Brain Energy is adjusted upward in box 608 to indicate replenishment. If the IMWL score is in the mid-range (greater than z-score_lo and less than z-score_hi), then no change is made to Total Brain Energy.

Box 610 and Box 612

Box 610 compares the Total Brain Energy Score (Box 608) to a TBE threshold. In some embodiments, the TBE threshold is a percentage of an average Total Brain Energy Score of healthy individuals at rest 102. If the Total Brain Energy is less than the TBE threshold, as to potentially trigger a concussion symptom or cause harm or regress their recovery, then an alarm is raised (at box 612) to the patient, warning them to rest and to cease the activity that is demanding a high mental workload from the patient.

Box 614 and Box 616

The patient is asked to calibrate the algorithm by doing a few minutes eyes open rest. The length of time depends on acquiring sufficient number of EEG epochs that are clean (i.e. no noise in the EEG signal) to achieve a representative distribution of IMWL scores of the brain at rest. Box 614 builds a probability distribution of IMWL scores of the person when it is known that they are well rested and have a large value for Total Brain Energy. Box 616 calculates the parameters of IMWL at rest distribution. In one embodiment, the mean and standard deviation of the IMWL rest distribution is calculated. These parameters are used by Box 606 to determine if the IMWL score is high enough to deplete Total Brain Energy or low enough to replenish Total Brain Energy.

FIG. 7 illustrates a data processing architecture schematic 700, in accordance with some embodiments.

FIG. 7 provides more details of the data processing, for example, that occurs in FIG. 6. Starting at box 702, a machine learning model is customized for a given patient. The model makes predictions of Instantaneous Mental Workload. One method of obtaining a customized machine learning model is to use supervised learning methods of labelled EEG data of the patient while they perform mental tasks. The system presents a number of mental tasks for the patient to complete while their EEG data is recorded. One way of training the model for the patient is as follows: the patient is asked to complete multiplication tasks of increasing difficulty. Ten minutes of EEG data is recorded while a patient solves simple multiplication problems of multiplying two single digit numbers. In the next ten minutes the patient is asked to complete intermediate level difficulty by solving the multiplication of two 2-digit numbers and finally another ten minute recording of the highest level of difficulty by solving multiplication of two 3-digit numbers. After each ten minute session the patient is asked to report the level of their mental workload. One way is to use a subjective scale called NASA Task Load Index (TLX). Another method can be to measure other physiological measures of mental workload. The level of mental workload is the dependent variable or the label for each section of EEG data. There are four levels of increasing levels of mental workload, beginning from rest all the way up to multiplying two 3-digit numbers, which covers the spectrum of mental workload that a patient may experience and hence increase the accuracy of predicting instantaneous mental workload. In addition, the patient is asked to rest for 5 minutes with their eyes open before the problem solving tasks and when the patient is mentally rested.

The above steps provide labelled data that can be used to train and validate machine learning models. The steps of machine learning are described as follows:

1) Preprocessing—The data is cleaned and pre-processed. In the case of EEG, cleaning and pre-processing includes removing sources of biological noise (such as eye movement, blinking, muscle noise). A classifier is used that is specific to the electrode and EEG device type. In one embodiment, an epoch is deemed to be clean when its variance is below a certain threshold and the maximum absolute value of the first derivative of the raw signal is below a threshold. Also, sources of environmental noise such as 60 Hz AC power line electromagnetic interference need to be filtered out. AC powerline noise is present when near household AC power. Preprocessing uses filtering of the raw EEG signal such as low pass, high pass, and notch filters. After preprocessing the raw EEG, the data is called “clean EEG”.

2) Feature Engineering—This is the mathematical transformation of the clean EEG into features. In frequency domain features, a Fast Fourier Transform using the Welch method is applied to one second epochs of cleaned EEG data. Other methods of calculating power spectral density, such as, for example, wavelet spectral analysis. This provides an EEG spectrum of the power in the clean EEG signal in microvolts² per Hz. The EEG spectrum is used to calculate the EEG bands such as delta, theta, alpha and beta. An example of time domain feature is the variance of a one-second epoch. Example of non-linear features are: entropy, Hurst component and Hilbert transform. Feature engineering can also combine information across multiple electrodes and or other features.

3) Feature selection—Often a long list of features are engineered. These features need to be reduced to a set of features that have minimal collinearity and that have high predictive value. One method of reducing collinearity is to apply Principal Components Analysis (PCA) or by removing features that are highly correlated. One method of feature selection is to use Random Forest feature importance. Numerous Decision Trees are created from the labelled data. The importance of each feature is determined by counting the number of times it is used in prediction across the numerous trees that were created using the labelled data.

4) Model Selection—Numerous criteria are applied to select a machine learning model. Usually the selection of the model is based on the judgment of the data scientist. Models can be classification (predict categories) or regression (predict a continuous value). In one embodiment, self-reported NASA TLX scores are used to label sections of EEG while the brain is respectively: at rest, simple mental task, intermediate mental tasks and difficult mental tasks are used as the continuous value dependent-variable for linear regression. Models need to be accurate, make quick predictions, and are generalizable and reliable even in the face of data it has not encountered before (i.e. avoid overfitting).

5) Training of the Model and validation—one method is to use cross-validation where the data is divided into k (in some embodiments, k=10) separate folds. where each fold is a different set of training and test data. The training data is separate from the test data in each fold. A model is trained using the training data. The trained model is used to make predictions with the test data. The best models will have the best accuracy across the k sets of test data as well as the lowest variance in accuracy across the k folds.

6) Prediction—After a model is validated it can be used to predict values of interest. In the present application, Instantaneous Mental Workload (IMWL) is the predicted value.

The aforementioned steps serve to create a machine learning model including the Calibration EEG Data which serves as input for a number of outputs.

Continuing with box 702, an output to be calculated is the patient Alpha Frequency (IAF). This is accomplished by analyzing the EEG spectrum to determine the peak within the alpha EEG band as well as the start (lower bound) and end of the alpha band (upper bound) that is specific to each patient. In this way the EEG bands can be customized per patient. This approach is described in the document “EEG alpha and theta oscillations reflect cognitive and memory performance: a review and analysis” by Wolfgang Klimesch Brain Research Reviews 29 1999 169-195. The EEG bands of theta, alpha and beta are set as follows: theta 4 Hz to lower bound of the IAF, alpha is the lower bound to the upper bound, and beta is the upper bound of the IAF to 30 Hz.

Continuing at box 702, the EEG features are calculated per epoch of EEG data. As an example, frequency domain features like the EEG bands customized using IAF are calculated. Other features such as time domain and spatial domain may also be calculated. Next a regression model such as linear regression or Decision Tree Regression is determined by using the labelled EEG data as described in the paragraph above. The regression model and its parameters (e.g. coefficients and intercept) is another output of the Training of the Models (box 702). Finally, the trained regression model is used to predict the Instantaneous Mental Workload of each epoch of the Eyes Open Rest session that was recorded from the patient. The parameters of this distribution (e.g. mean and standard deviation of the distribution of predicted Instantaneous Mental Workload predictions) is the final output of the Training of the Models (box 702).

Both the Training of Models (box 702) and Instantaneous Mental Workload score predictor (box 704) need Signal Processing Parameters (box 706) used in preprocessing as well as the parameters specific to different EEG devices (box 708).

In box 706, the signal processing parameters are defined as follows:
FS min is the range of acceptable sampling rates from the EEG device. If the sampling rate falls out of this range then an error flag is raised.
Good signal ratio is the percentage of epochs that have clean EEG data. An error is raised if this falls below a certain ratio.
Cut-off frequency is the highest frequency used in the EEG spectrum (e.g. 64 Hz). Electrodes are the scalp locations where EEG electrodes are placed (e.g. A1, AF7, Fpz, AFB, A2).
Finally, the parameters and type of filtering needed to filter the raw EEG data (e.g. low-pass filter at 40 Hz cut-off, high pass filter at 1 Hz cut off, notch filter at 60 Hz).

Box 708 lists parameters related to EEG Device Type. The device name and model, are respectively, the manufacturer and model of the EEG device. Sampling frequency is the rate that analog EEG voltages are sampled to be converted into digital samples. Window length is the size of the epoch in seconds or number of samples. Window overlap is the percentage of overlap from one epoch to the next.

At box 704, the outputs from box 702, along with the parameters from box 706 and box 708, serve as inputs to calculate a number of outputs, such as one or more meter scores and a signal quality error. Examples of meter scores are listed in box 710; these include an Instantaneous Mental Workload score (referenced in box 602 of FIG. 6), a Total Brain Energy Measure meter score (referenced in box 606 of FIG. 6), and a Raise Alarm (box 610 of FIG. 6). Examples of error flags are shown in box 612. These include errors when the sampling rate is too low, or if the signal quality is too low.

The systems and methods disclosed herein include intake of patient data, which comprises a baseline EEG (also called Calibration EEG) and revised scores (including balance measure, injury mechanism, patient history and current symptoms/triggers). A physical or cognitive session starts, and is paused to provide a patient with one or more alerts when the patient is approaching either a cognitive or a physical overexertion. The systems and methods disclosed herein facilitate interaction with a patient by allowing the patient to input symptoms and suspected triggers. Reports of data analysis are provided to the patient.

Brain Break Alert

The systems and methods disclosed herein prevent mental over-exertion and cognitive fatigue in concussion patients using EEG data. An algorithm differentiates between rest, cognitive engagement, high mental workload and cognitive fatigue in concussion patients using frequency-based linear, time-domain and non-linear features.

When a concussion patient sits down to perform a cognitive task (for example, homework), they place the EEG headset on their head and launch a customized application on a mobile device (for example, a mobile phone, a tablet, wearable technology). When the patient begins to overexert themselves cognitively, they will receive a brain break alert that suggests they rest their brain. If they receive a symptom (or trigger) during the activity, they log the symptoms and the task is paused so they can rest. The systems and methods disclosed herein allow patients to return to work progressively, so they do not regress during their recovery process. The patient is able to track their recovery sessions over time, learning what type of activities strain their brains and which do not.

A combination of frequency-based linear, time-domain and non-linear EEG features, along with blink frequency and duration are used to differentiate between rest, cognitive engagement (healthy), high cognitive workload and fatigue in concussion patients. The specific parameters used to determine this brain states are refined to the patient based on calibration data collected at the start of each cognitive session. This allows the brain alert to be specific to the patient's brain as they recover or regress during their recovery process. When high cognitive workload or fatigue is detected by the algorithm, the patient will receive an alert on their mobile application suggesting that they take a cognitive break.

Body Break Alert

The systems and methods disclosed herein prevent physical over-exertion in a concussion patient by using the patient's heart rate data. Similar to cognitive over-exertion, physical overexertion can be detrimental to concussion patients. A physical overexertion algorithm (as illustrated in the example shown in FIG. 6) alerts the patient when they are over-exerting themselves physically, based on heartrate taken from a device that measures the heartrate of the patient.

A resting heart rate and max heart rate before symptoms of a patients are established. Clinical recommendations state that exercise heartrate should be less than 70% of this max for a concussion patient. This maximum limit is calculated by the algorithm and the patient is able to exercise up to this point before receiving an alert on their mobile application.

Restoration for a Concussion Patient

The systems and methods disclosed herein provide neurofeeback-based meditation to restore cognitive function post-brain injury. The system and methods use real-time EEG to provide feedback to help concussion patients rest and meditate.

Concussion Diagnosis and Return-to-Play Tool

The systems and methods disclosed herein provide a tool for healthcare providers to assess the improvement of a patient. Combined EEG, balance score and neurocognitive assessments are used to improve return to life (sport, work, school) decisions being made by doctors.

Risk Assessment

As stated above, the use of dry sensors precludes the need for skin abrasion, which introduces the risk of infection and pain for the subject. Potential risks to the participants are very minimal. There is no psychological or social risk; all participants have their confidentiality maintained, in accordance with local privacy legislation. The nature of the data collected does not pose a social risk even if identified data were accidentally made public, as the data comprises bioelectric signal measurements, generally in a graph form that contains information about electrical brain signals.

Box 602—Linear and Non-Linear Models

Implementation A of Instantaneous Mental Workload Score (IMWL)—Linear Regression Model based on Frequency Domain Features

IMWL=b1*EEGband1+b2*EEGband2+ . . . bn*EEGbandn+c

Where:
IMWL=Instantaneous Mental Workload Score range from 0 to 100 where 100 is the maximum possible level of mental workload;
bi=coefficients of the linear model that can be learned by example from linear regression;
EEGbandi=the power spectral density (PSD) of an EEG signal (example: alpha EEG band is the power in an EEG signal from 7.5 Hz to 12 Hz); and
c=constant

Implementation B of Instantaneous Mental Workload Score (IMWL)—Non-Linear Model based on Regression Decision Tree of Frequency Domain Features.

Implementation C of the IMWL—all features are available to be used, such as, for example, EEG band features, frequency domain features, time-domain features, non-linear features, among other features that may be captured, such as eye motion, blinking, muscle tension, accelerometer, gyroscope, magnetometer, heart rate, hear-rate variability, and the like. In this implementation, IMWL=b1*EEGf1+b2*EEGf2+ . . . +bn*EEGfn+c; where EEGfi is a feature calculated from a signal epoch of clean EEG data and or features of other physiological measurements that include but not limited to body motion, balance, eye motion, blinks, muscle activity, heart-rate, heart-rate variability, and the like.

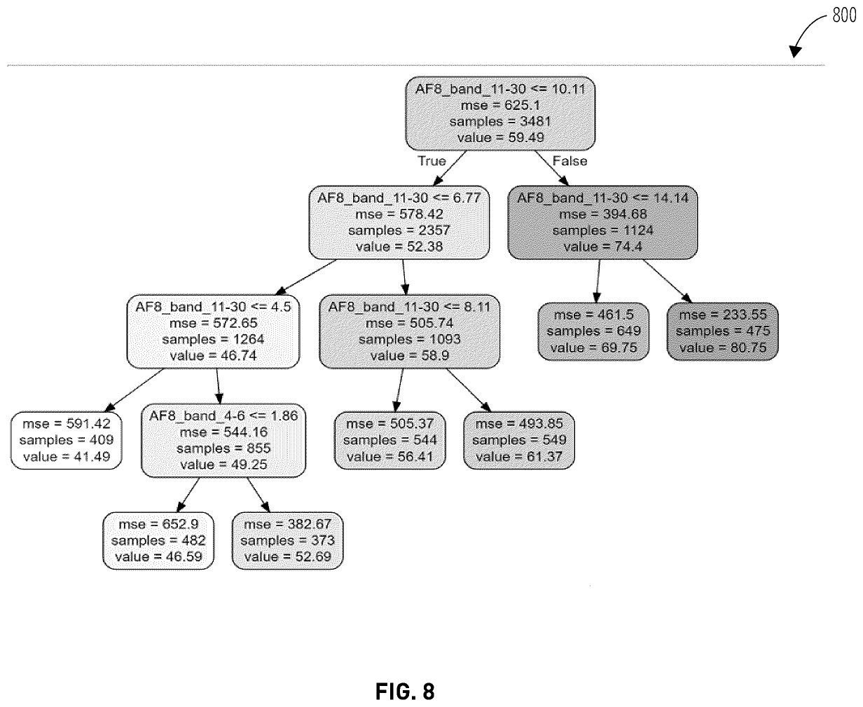

FIG. 8 shows an example of a Regression Decision tree 800 used to predict IMWL. The values in the leaves of the tree are the IMWL. The variables show the EEG electrode and the personalized EEG band for this patient. As an example, AF8_band_11-30 is the PSD of the EEG signal from electrode AF8 in the range of 11 to 30 Hz.

FIG. 9 is a flow diagram of a method 900 to train a machine learning model, in accordance with some embodiments. The method 900 is performed by the BBA module 224 of FIG. 2 in conjunction with a user device, for example, the user device 206 of FIG. 2. For example, the ML model of the BBA module 224 is trained by first having the patient 232 perform one or more tasks, while the patient is wearing the EEG headset 202 and while the EEG data captured by the EEG headset 202 is sent to the system server 210. In some embodiments, the one or more tasks form an engagement protocol for the patient 232, where the engagement protocol is defined to assess the patient's 232 brain activity, and to train the ML model customized to the patient 232.

Different combinations of cognitive tasks are chosen as a part of the engagement protocol to exercise different functions of the brain such as visuospatial tasks, arithmetic, logic puzzles, executive function and memory, for example. Rest is included as one of the tasks in order to present machine learning models with rest and task activities, both of which are needed to capture the full range of cognitive functions, and especially to model the gradient of easy (i.e. rest) to increasingly demanding cognitive functions. In some embodiments, as a part of the engagement protocol, the patient is asked to perform seven activities or tasks of varying difficulty while their EEG data is recorded. In one implementation, the seven activities are rest, three levels of increasing difficulty of visuospatial tasks (e.g. Raven's Progressive Matrices logic puzzles), and three levels of increasing difficulty in arithmetic such as multiplication comprise the seven tasks. Further, as a part of the engagement protocol, the patient's self-reported cognitive exertion and fatigue is received as an input, for example, via the GUI 230, after each task.

In a specific, non-limiting example of the engagement protocol, the first task is rest. The patient sits with eyes closed or open for two minutes to help the electrodes stabilize their impedance values and to determine the different EEG signatures present while eyes are closed. Then the patient sits for several minutes resting and using as little cognitive effort as possible with their eyes open. Rest is done before the problem-solving tasks and when the patient is mentally rested. Next, the patient solves as many multiplication questions as they can complete in 5 minutes of simple multiplication problems of multiplying two single digit numbers. In the next task the patient is asked to complete intermediate level difficulty by solving the multiplication of two 2-digit numbers. Another recording of EEG data is taken in the final multiplication difficulty level, the patient solves as many multiplication problems between two 3-digit numbers for 5 minutes—highest level of difficulty for multiplication. In addition, three sessions of increasing level of difficulty of logic puzzles are also completed by the patient. After each task, the patient/patient is asked to report the level of their level of cognitive exertion or mental workload that was used to complete each task. In some embodiments, a subjective scale such as a NASA Task Load Index (TLX), which is a well known scale to measure perceived level of cognitive exertion, is used. The level of cognitive exertion is the dependent variable or the label for each section of EEG data. There are seven levels of cognitive exertion, beginning from rest, multiplying numbers and solving logic puzzles, which covers the spectrum of cognitive exertion across three different ways of using the brain and hence increasing the accuracy of predicting instantaneous mental workload.

In some embodiments, the above or similar activities may be administered to the patient by another person, such as a caregiver or a healthcare professional via the GUI 230 of the app 228 on the user device 206. In some embodiments, the patient 232 is able to self-administer the engagement protocol using the GUI 230 of the app 228, which includes instructions for the user to perform an activity or a task, and to provide an assessment of the mental workload associated with the task as input to the app 228. In an example of automated administration of engagement protocol, a patient, for example, the patient 232 is asked to rest and or perform a cognitive activity or task while their EEG data is recorded by the EEG headset 202. The automated Engagement Protocol (AEP) includes a set up phase in which the patient 232 first generates a baseline of rest with one period with eyes closed and with another period with eyes open. After each period of rest, the patient 232 is asked to report one or more of the following measurements:

MENTAL ENERGY: On a scale of 1 to 10 how much mental energy did you expend while resting? If you were a 10, you are super fresh, ready to write a hard exam, 1 is completely drained.

COGNITIVE ENGAGEMENT: On a scale of 1 to 10 how mentally engaged were you while resting, where 1 is low level of engagement and 10 is the highest level? In other words, how much did you have to concentrate on this task on a scale from 1-10. In some embodiments, NASA TLX scale is used.

COGNITIVE FATIGUE: On a scale of 1 to 10 how mentally tired were you while resting, where 1 is extremely mentally tired and 10 is the highest level? In other words how drained do you feel after completing this task.

After the rest, the patient is asked to complete different levels of difficulty of several cognitive activities. In one implementation three difficulty levels for a given type of a cognitive activity are used, however, more levels can be used. So as an example of one level of cognitive task, the patient is asked to perform a multiplication activity, such as a multiplication of 2 digit numbers. In some embodiments, the app 228 may demonstrate via the GUI 230 how to perform the task, for example, using a piece of paper and pen or using the GUI 230 screen to solve multiplication problems. The app 228 may remind the patient that it is important that the patient attempt their best while completing these activities, in order to generate an accurate set of EEG data that reflects a wide range of cognitive engagement and fatigue. The app 228 can also be used to determine how busy the patient is and if they are performing well by determining if the answers to the questions they are asked to complete are correct. The EEG headset 202 can also be configured to capture physiological data about the amount of eye movement, where the lack of eye movement may indicate focus. An accelerometer/gyroscope/magnetometer usually found in smartphone type user devices 206 may also be used to determine if the patient/user's head posture indicates focus. After each cognitive task, the user is asked the following questions:

MENTAL ENERGY: On a scale of 1 to 10 how much mental energy did you expend while completing the task? If you were a 10, you are super fresh, ready to write a hard exam, 1 is completely drained.

COGNITIVE ENGAGEMENT: On a scale of 1 to 10 how mentally engaged were you while completing the task, where 1 is low level of engagement and 10 is the highest level? In other words, how much did you have to concentrate on this task on a scale from 1-10

COGNITIVE FATIGUE: On a scale of 1 to 10 how mentally tired were you while completing the task, where 1 is extremely mentally tired and 10 is the highest level? In other words how drained do you feel after completing this task.

In addition, the user is asked to complete the self assessment of their cognitive fatigue/mental workload using NASA TLX scores in two parts: with a number of pairwise comparisons of different cognitive aspects, and the ratings of each aspect.

The patient 232 is presented definitions of each aspect of the workload (activities) they are presented with, and perform a comparison for each set of these aspects, where the patients identify which aspect was a more important contributor to the mental workload the patient felt while completing the task. Thereafter, the patient 232 inputs a rating of the mental workload on a scale of 1 to 100, and/or NASA TLX scale. In some embodiments, the scale uses inverted ratings, where 0 is rated as good and 100 is rated as poor.

The BBA module 224 administers activities to the patient 232 via the GUI 230 on the user device 206. The patient 232 follows the instructions on the GUI 230 to perform the activities, and provide as input, the mental workload perceived by the patient 232 while performing or after performing the activities.

At step 902, the method 900 captures EEG data at the EEG headset 202. The EEG data includes electric signals captured by electrodes of the EEG headset 202 in contact with the head of the patient 232. At step 904, the method 900 sends the captured EEG data to the user device 206, which receives the EEG data at step 906, sends the EEG data to the system server 210 at step 908, and the system server 210 receives the EEG data at step 910. In some embodiments, steps 902-910 are performed while the patient 232 is performing the one or more activities, or after performing the one or more activities. In some embodiments, the steps 902-910 are performed at predefined intervals, or instantaneously.

At step 912, the method 900 pre-processes the EEG data. In general, unprocessed EEG data or raw EEG data is cleaned to remove noisy epochs, to yield unfiltered clean EEG data which represents true brain signals (without noise), and such unfiltered clean data is usable to calculate frequency domain features. A filter (e.g. IIR or FIR) is applied to the unfiltered clean EEG data to yield filtered clean EEG data, which is usable for time domain or non-linear features.

As a part of pre-processing, raw voltage values of EEG data from one or more electrodes from a the patient's head may also be converted into digital samples. The digitized EEG signals are processed, and cleaned, by filtering the EEG data and removing noise and movement artifacts. In some embodiments, one or more noise classifiers as known in the art are used identify and remove noise. For example, a classifier specific to each electrode in the EEG headset is trained using supervised machine learning of hundreds of known examples of brain signal, such as blinks, eye motion and noise. The noise classifier determines for each second of EEG data if it is brain signal, a blink or noise. Noise can be biological or environmental, for example, eye movement, blinks, muscle tension, motion of electrodes across skin (vibration or mechanical shock), EMI such as 60 HZ or other sources of radio interference, and electrode depolarization (static discharge, change in charge, sudden changes in impedance). Features are calculated per epoch such as maximum slope, maximum amplitude, range, frequency domain features, flat-lining (long period of no change in voltage value), delta EEG band power, fractal dimension (self-similarity). In some embodiments, supervised machine learning method, such as a decision tree, is applied to raw EEG data. In addition, the noise classifier also determines if there has been a lost packet(s) in the transmission of EEG data from the headband to the smartphone. The noise classifier also determines if the headband has been removed from a patient/user's head.

In some embodiments, noise is compensated using a noise compensation method that predicts the IMWL during blinks and other sources of biological noise is presented in which the IMWL for a window with a blink is calculated by averaging previous clean windows in a 4-second input epoch (although intervals other that 4 seconds may also be used), broken down into normal 1-second windows with 50% overlap. For example, biological sources of noise such as blinks, eye movement, muscle contractions or any source of other noise (e.g. electrode movement, electrostatic discharge, environmental (EMI etc.) obscure the frequency domain features captured to predict IMWL. Instantaneous mental workload (IMWL) is calculated while patients are engaged in a task on clean brain signal. During a task, various sources of noise such as eye motion, poor electrode connections, head motion, or blinks can reduce signal quality and results in an artificially low estimation of cumulative mental energy expenditure over time. The goal of IMWL noise compensation method is to approximate a reasonable IMWL for epochs that contain task-relevant artifacts such as blinks, but not to compensate for task irrelevant artifacts such as instrumentation noise or head motion.

The noise-compensation algorithm receives 4 seconds of data every 500 ms from the app 228. This 4-second segment is windowed into six 1-second windows that overlap each other by 50%. IMWL is calculated for the last 1-second window in the input data epoch. If this window is clean EEG signal, then the IMWL for this window is returned to the app 228, which is then used to determine the user-specific Brain Energy used for that epoch. If the window is not clean, but contains a blink or noise, an IMWL equal to the mean of all clean windows in the 4-second input epoch is returned. If there are no clean windows in the epoch, then no IMWL is returned and there is no Brain Energy used for that epoch. If the last window is a non-blink artifact, or if there are no clean windows in the input segment, then no Brain Energy use occurs, or a default value of Brain Energy Used is provided such as mean BEU for that user. In some embodiments, the app 228 generates an alert to the patient when the amount of data lost due to either lost data either due to poor electrode signal quality or for excessive Bluetooth packet loss exceeds a predefined threshold.

In some embodiments, to reduce the amount of redundant data transfer, epoch size is increased from 4 seconds to 11 seconds, the epoch transfer rate to the app 228 is decreased from 1/0.5 seconds to 1/10 seconds, and IMWL was always taken as the average IMWL over all clean windows within an epoch. Noise compensation is then implemented in the app 228 in a similar fashion as discussed above. If there are any clean windows in an epoch, then IMWL and Brain Energy used value will be returned. If there are no clean windows in an epoch, then the app 228 uses the average of up to the last 6 Brain Energy used values to determine the new BEA. If there are no prior clean epochs, then the app 228 uses a default Brain Energy used value to determine the new TBE which is 0.5*BEM (brain energy multiplier). For further noise compensation, all default Brain Energy used values are ignored when calculating the noise-compensated Brain Energy used value.

Pre-processing further includes feature engineering, which is the mathematical transformation of the EEG data into features, which are calculated for each epoch. The features of the EEG signal can be frequency domain, time domain or spatial or non-linear. The features are neurophysiological markers of mental workload or cognitive exertion specific to a patient. In frequency domain features, a Fast Fourier Transform (FFT) using the Welch method is applied to one-second epochs of unfiltered clean EEG data, which provides an EEG spectrum of the power in the unfiltered clean EEG signal in microvolts per Hz. The EEG spectrum is used to calculate the EEG bands such as delta, theta, alpha, and beta, and the EEG bands are customizable for each patient.

In some embodiments, the EEG bands use the patient's individual alpha frequency (IAF). The IAF is determined by analyzing the EEG spectrum to determine the peak within the alpha EEG band as well as the start (lower bound) and end of the alpha band (upper bound) that is specific to each patient. The IAF may be approximated to by calculating the lower bound as peak alpha−3, and the upper bound as peak alpha+2. Separate IAF is also calculated for each electrode, and the EEG bands are customized per patient for each electrode. In some embodiments, the EEG bands of theta, alpha and beta are set as follows: theta 4 Hz to lower bound of the IAF, alpha (i.e. IAF) is the lower bound to the upper bound, and beta is the upper bound of the IAF to 30 Hz.

Further, the features can be linear, for example, time domain feature is the variance of a one-second epoch, or non-linear, for example, entropy, Higuchi's fractal dimension, Lempel-Ziv complexity, Hurst exponent and Hilbert transform and the like. The Hilbert transform may be used for calculating features related to phase, such as phase locking value. Features of spectral coherency, spectral coherence, and spectral connectivity between pairs of electrodes may also be calculated. Electrophysiological signals related to cognitive fatigue, for example, blinks can also be detected as follows. Each eye has an electrical potential across the cornea to the retina, and when the eye moves or blinks, this electrical field changes and can be detected by frontal EEG electrodes. Features can be extracted from the shape of these eye-based signals and be used to infer the rate of blinking, and the duration of the blink, blink amplitude, eye closure time, and eye re-opening time.

After pre-processing a step 912, at step 914, after completion of one of the one of more activities by the patient 232, the method 900 receives an input from the patient 232 on the user device 206. The input is the patient's assessment of the patient's mental workload or cognitive exertion, representing the brain energy used in performing the activity. The patient's assessment could be on any scale, for example, a ‘low’, ‘medium’ or ‘high’, or a scale of 1-10, or other similar scales known in the art. In some embodiments, a subjective scale known as NASA Task Load Index (TLX) is used as the scale by the patient 232.

At step 916, the method 900 sends the input from the user device 206 to the system server 210, which receives the input at step 918.

At step 920, the method 900 trains the ML model to predict the IMWL based on the input of the mental workload and the EEG data. In some embodiments, the ML model is one or a more of a linear regression model, decision tree regression model, partial least squares model, another regression model, or a classifier model. In some embodiments, the ML model is a linear regression model due to the ability to predict IMWL on a continuous scale. The method 900 provides the training data including the EEG data for the patient for the duration the patient is performing an activity/task, for example, pre-processed using one or more techniques discussed above, and the patient's input on the mental workload (cognitive exertion) spent to accomplish the activity/task.

In one embodiment, as discussed earlier with respect to FIG. 7, a linear regression model based on EEG features is used as the ML model and is defined as:

IMWL=b1*EEGf1+b2*EEGf2+ . . . bn*EEGfn+c (Equation 1),

where:

IMWL=Predicted Instantaneous Mental Workload range from 0 to 100 where 100 is the maximum possible level of mental workload;

bi=coefficients of the linear model that can be learned by example from linear regression;

EEGfi=a feature an EEG signal (example: alpha EEG band is the power in an EEG signal from 7.5 Hz to 12 Hz); and

c=constant.

In some embodiments, all features available are used at first, and then a subset of features that are accurate in predicting IMWL are selected. The ML model is trained, the coefficients and the constant values are arrived at, and the trained ML model is validated. At step 922, the method 900 tests, at the system server, the ML model trained by steps 902-920. In some embodiments, the method 900 repeats one or more steps of the method 900 to further train the ML model till such time a desired accuracy is obtained. In other embodiments, multiple ML models are trained using the training data, and the most accurate model is selected.

For example, cross-validation is used where the data is divided into multiple (e.g., ten) separate folds where each fold is a different set of training and test data. The training data is separate from the test data in each fold, and an ML model is trained using the training data. The trained ML model is used to make predictions with the test data, and accuracy of the predictions is calculated as the difference from the actual cognitive exertion and the prediction calculated using root mean squared error, although other known methods to calculate accuracy. The ML model having the highest average accuracy across the ten sets of test data as well as the lowest variance in accuracy across the ten folds is selected.

While in some embodiments, the EEG data is sent via the user device 206, for example, via steps 906 and 908, in some embodiments, the EEG data may be sent directly from the EEG headset 202 to the system server 210, such that the EEG data is received at step 910 from the EEG headset 202.

In some embodiments, the features for training the model are selected for a high predictive value and/or minimal collinearity. In some embodiments, the features are calculated across four electrodes of the EEG headset. Collinearity may be reduced by applying principal components analysis (PCA), or by removing features that are highly correlated. In some embodiments, the Random Forest method is used to calculate the importance of each feature, multiple decision trees are created from the labelled data, and the importance of each feature is determined by counting the number of times the feature is used in prediction across the decision trees that were created using the labelled data, for example. In some embodiments, only those features that are identified as important in feature selection are used to train the ML model and used for predicting IMWL.

In some embodiments, a machine learning (ML) model may be selected based on different criteria. Models can be classification oriented (predict categories) or regression oriented (predict a continuous value). In some embodiments, scales/scores such as NASA TLX are used to label sections of EEG data while the brain is respectively at rest, performing easy cognitive tasks, performing intermediate cognitive tasks, and performing difficult cognitive tasks. In such embodiments, the NASA TLX (or any other scale) scores reported for each task are used as the continuous value dependent-variable for training the regression classification model. Desirable factors for selecting an ML model include accuracy, speed (i.e., making quick predictions), generalizable, and reliability with new data, that is, the ML model avoids overfitting. In some embodiments, one or more of linear regression, decision tree regression, and partial least squares are used.

FIG. 10 is a flow diagram of a method for generating a brain break alert, in accordance with some embodiments. The method 1000 is performed by the BBA module 224 of FIG. 2 in conjunction with a user device, for example, the user device 206 of FIG. 2. The BBA module 224 includes an ML model trained by the method 900 of FIG. 9 using the patient data 222 (EEG data) of the patient 232, and therefore, the ML model is customized according to the patient 232. The trained ML model includes all of the parameters needed by the model to predict IMWL, BEU and BBA, such as coefficients to determine IMWL: b1, b2, . . . bn, c; task_mean of IMWL distribution; std_task of IMWL distribution; BEU used to reach BBA is arbitrarily set as 300 as the standard in this example, and 300 would be used for all patients as we are trying to standardize each patient BEU to reach BBA as 300; and BESF. The BEI is set as 3600, but may be set to another number.

The ML model customized to the patient 232 is used to predict IMWL for the patient 232, while the patient is wearing the EEG headset 202 and performing one or more tasks. It is theorized that such a customized ML model would be highly accurate and/or reliable for predicting the IMWL for the patient 232 based on the EEG data of the patient 232.

At step 1002, the method 1000 captures EEG data of the patient 232 by the EEG headset 202, and at step 1004, sends the captured EEG data to the user device 206, which receives the EEG data at step 1006, and at step 1008, sends the EEG data to the system server 210. While in some embodiments, the EEG data is sent via the user device 206, for example, via steps 1006 and 1008, in some embodiments, the EEG data may be sent directly from the EEG headset 202 to the system server 210, such that the EEG data is received at step 1010 from the EEG headset 202.

At step 1010, the method 1000 receives the EEG data at the system server 210, and at step 1012, the method 1000 pre-processes the EEG data at the system server 210. At step 1014, the method 1000 determines IMWL based on the EEG data (or pre-processed EEG data) by the ML model of the BBA module 224 on the system server 210.

At step 1016, the method 1000 determines the brain energy used (BEU) from IMWL. According to some embodiments, the BEU is a cumulation of IMWL (or a function thereof) for a duration of time while the patient 232 is engaged in an activity, during which generating a brain break alert (BBA) is desirable. At step 1018, the method 1000 determines the brain energy available (BEA) to the patient 232. According to some embodiments, the BEA at a given time is proportional to a difference between an initial brain energy (BEI) of the patient 232, and the BEU of the patient 232 till that time. In some embodiments, the BEI is customizable quantity, and in some embodiments, the BEI is set to 3,600 units.

In some embodiments, the BEU is a summation of the IMWL for each second from the start, up to the time at which BEU needs to be determined. In some embodiments, the BEU is a summation of IMWL for adjacent predefined periods of time. In some embodiments, the BEU is a summation of a running average of IMWL for adjacent predefined periods of time. In some embodiments, the BEU is a summation of IMWL for adjacent overlapping predefined periods of time, averaged to account for the overlap. In some embodiments, the BEU is determined based on more complex operations on the IMWL, as discussed below.

It is theorized that as the patient 232 performs an activity, more brain energy is used, and consequently the BEA is reduced, and an alert for the patient 232 to take a break should be generated before the BEA falls below a threshold value, that may be predefined and/or customizable (for example, as shown in FIG. 5).

Returning now to the operation of the method 1000, the BEA is tracked over time using the method 1000, for example, by continually iterating steps 1002-1018, or cumulatively determining the BEU or the BEA by iterating steps 1010-1014. At step 1020, the method 1000 determines that the BEA satisfies a brain break alert threshold (BBAT), for example, when the BEA becomes equal to or lower than the BBAT. Upon determining that the BBAT has been satisfied by the BEA, at step 1022, the method 1000 sends an instruction to generate a brain break alert (BBA). In some embodiments, the method 1000 sends the instruction from the system server 210 to the user device 206, which, at step 1024, generates the brain break alert (BBA) 234 via the GUI 230. In some embodiments, the method 1000 sends the instruction from the system server 210 to the EEG headset 202, which, at step 1026, generates the brain break alert (BBA) via devices included therein, such as a vibrating device or an audio device.

Several other methods may be used to determine the BEU using the IMWL determined by the ML model (step 1016). Use of such methods may require additional or modified steps for the method 900 of FIG. 9 and/or for the method 1000 of FIG. 10, as discussed below.

In some embodiments, a method 1 to determine the BEU from IMWL and generate the brain break alert (BBA) includes determining a distribution of IMWL of rest for the patient 232. A probability distribution function of the IMWL during rest is determined by using the IMWL prediction for each one second epoch of the individual at rest. The ML model has been pre-trained using NASA TLX scores having a range from 1 to 100, where 100 is the highest mental workload. A z-score is used to normalize the IMWL score with respect to rest and provides a measure of cognitive exertion that is relative to a person's rest. The z-score is a statistic that measures the distance in standard deviations of a sample against a reference distribution. This z-score is known as brain_change, which is IMWL normalized as a z-score. The reference distribution is the IMWL score of the recording of eyes open rest of the patient. One standard deviation above the mean of the reference distribution of IMWL establishes the threshold for z-score_hi (i.e., z-score_hi=+1). A single value of IMWL needs to be greater than z_score_hi to contribute to a decrease in brain energy available (BEA). IMWL values are transformed into brain_change using the following formulae.

$$z\_score = \frac{IMWL - µ}{σ}$$

$$brain\_change = \{ \begin{matrix}z\_score & if z\_score > + 1 \\ 0 & if z\_score < + 1\end{matrix}$$

where:

IMWL=Instantaneous Mental Workload

μ=Mean of the distribution of IMWL during eyes open rest

σ=Standard Deviation (SD) of the distribution of IMWL during eyes open rest

brain_change=IMWL normalized as a z-score

BEA is the amount of mental energy that a patient has available to perform cognitive activities. The BEA for the patient 232 at any given time while the patient is performing an activity is determined as:

BEA=BEI−BEU,

where

BEI is a maximum amount of brain energy that the brain can hold; and

BEU is brain energy used by the brain to do cognitive work from the start of an activity by the patient 232 up to the given time.

In the method 1, each time the patient uses mental effort above rest state, their BEA is depleted in proportion to their brain_change as discussed above. If the IMWL score is greater than the threshold z-score_hi, then the patient's cognitive exertion is deemed high enough to deplete the patient BEA, and the BEA is adjusted downwards in proportion to the level of IMWL above z-score_hi. The formula for BEU across k epochs is:

$$BEU = \frac{{\sum }_{i = 1}^{k} brain\_change ( i )}{max\_MBC}$$

According to some embodiments, BEI is set to a default of 3600 units of brain energy. Therefore, BEA after k epochs is determined by:

$$BEA ( k ) = 3600 - \frac{{\sum }_{i = 1}^{k} brain\_change ( i )}{max\_MBC}$$

where:

BEA=Brain Energy Available

brain_change(i)=change in cognitive exertion in standard deviations from mean rest for epoch i

max_MBC=maximum mean brain change of the most challenging task done by the patient

k=The number of epochs in the EEG signal

epoch=length of time set of features are calculated across. 1 second is typical epoch length.

Further, for determining max_MBC, mean brain change is determined for each activity/task across k epochs that occurred during a task m by the formula:

$${MBC}_{m} = \frac{{\sum }_{i = 1}^{k} brain\_change {( i )}_{m}}{k}$$

and max_MBC is the highest MBCₘ across all of the tasks.

FIG. 6 illustrates the flowchart 600 that utilizes the method 1. As an example of how the BBA threshold is calculated, the BBA threshold is set to when an individual accumulates 20 minutes of cognitive exertion at the maximum level. The BBA threshold is the point where an individual maintains a constant effort of brain_change equal to the task during the Engagement Protocol that had the highest mean predicted brain change (i.e. max_MBC).

In this case the patient's max_MBC=3 and t=20 minutes, k=time/(epoch stride)=20 minutes*60 seconds/0.5 second=2400 epochs, max_MBC=3, and brain_change per epoch=3. Calculating accordingly:

$$BEA ( k ) = 3600 - \frac{{\sum }_{i = 1}^{k} brain\_change ( i )}{max\_MBC} \\ BEA ( 2400 ) = 3600 - \frac{{\sum }_{1}^{2400} ( 3 )}{3} = 3600 - 2400 \\ BBA Threshold = {BE}_{avail} ( 2400 ) = 1200$$

Here, it is assumed that the patient starts their day with 3600 units of brain energy, or BEI, which can be inferred from the self report of the patient during their Engagement Protocol based on the speed that the patient became fatigued while completing their tasks. In this example BEI is set to 3,600, however any other value can be used. BEI and BEU are adjustable for each patient based on their age, severity of their concussion or other factors as judged by their physician. In this example, the BBA threshold allows the patient a total sum of 20 minutes (2400 epochs) of maximal cognitive exertion for this day. BEA decreases as the day progresses and the patient exerts brain energy. As the above example shows, the patient exerted 2400 units of brain energy and was left with 1200 units as BEA. The BBA threshold in this example equals 1200 units. The result above shows that the BBA threshold for 20 minutes of sustained effort at max_MBC level will always be at 1200 based on 20 minutes regardless of the patient because BEA is scaled by the patient's own max_MBC. FIG. 5 shows a brain break alert (BBA) graph depicting depletion of the the BEA as the patient increases their mental workload. If the patient uses less than the maximal cognitive effort then the number of epochs and therefore the time to reach the Brain Break Alert will be longer than 20 minutes or 2400 epochs.

Table 1 shows the correlation that is achieved by method 1 algorithm's prediction of BEA compared to self-reported cognitive fatigue during the algorithm training tasks (i.e. Engagement Protocol). A high correlation score indicates high accuracy of the algorithm to predict the BEA, and all correlations are highly statistically significant (p-value <0.0001 probability that the correlation is 0). The correlations show that the strength of the relationship between BEA and fatigue, the p-values allow us to reject the null hypothesis that the two variables have no (i.e., 0) correlation. Table 1 shows that the customized algorithm approach works across ten patients used to test the method 1.

TABLE 1

| Participant | Correlation | p-value |
|---|---|---|
| VAK-1 | 0.87 | <0.0001 |
| VAP-1 | 0.79 | <0.0001 |
| VDM-1 | 0.86 | <0.0001 |
| VDM-2 | 0.55 | <0.0001 |
| VJEH-1 | 0.95 | <0.0001 |
| VKA-1 | 0.81 | <0.0001 |
| VLP-1 | 0.89 | <0.0001 |
| VLP-2 | 0.91 | <0.0001 |
| VME-1 | 0.82 | <0.0001 |
| VMK-1 | 0.56 | <0.0001 |
| VJH-1 | 0.79 | <0.0001 |
| VMC-1 | 0.34 | <0.0001 |

In some embodiments, a method 2 to determine the BEU from IMWL and generate the brain break alert (BBA) includes various alterations to the method 1, including alterations of feature values, adding individual beta frequency to training the ML model, use of a sigmoid function to smoothen brain energy, and normalize and scale brain energy. In some embodiments, the engagement protocol discussed above with respect to FIG. 9 is altered, including altering one or more parameters, such as using all eyes open tasks in training instead of just the first eyes open rest recording, removing eyes close rest data from the training pipeline, using log₁₀ of frequency domain power, and using relative power with respect to the 1-40 Hz band, for example, as discussed below, among others.

In the method 2, individual beta frequency (IBF) is added to the training of the ML model. Beta frequency is selected for predicting IMWL. The existing algorithms add power from all 6 Hz-wide bands between 16 and 45 Hz, in intervals of 2 Hz as features, then determine relative importance from a feature pool with many other features. The individual beta frequency (IBF) band is defined as the 6 Hz-wide frequency band that differs the most between rest and task for each user. It is theorized that customizing beta frequency bands increases the accuracy of the BBA module 224. For example, the beta band could be located with the same accuracy as the frequency resolution of the power spectral density estimate (the nearest Hz) instead of to the nearest 2 Hz. Further, reducing the size of the feature set decreases the probability that any of the important features found were important only by chance. For the reason that the Beta band is defined per electrode, determining individual beta frequency (IBF) before feature selection eliminates 10-11 features per electrode.

In some embodiments, the IBF is found using the relationship between each eyes open rest session and each task session, for example, by searching within the 12-35 Hz band. First, the average relative power for each frequency bin in the 12-35 Hz band is calculated for each window across clean epochs using the 1-35 Hz band, which is performed on each session in the training or engagement protocol, excluding the eyes closed rest sessions. Next, the average relative power for the 12-35 Hz band is calculated for each included session, and next, for each eyes open rest recording, using one eyes open rest as a baseline, the difference between relative power at rest and during each task is calculated and summed. This process is repeated for the second eyes-open rest, and yields a plot that shows the areas of the Beta band that consistently differ between rest and task. Using this plot, the IBF is determined as the 6 Hz region of the plot with the largest positive relative power, for each electrode. The process is repeated for the region with largest negative difference, yielding two bands per electrode using the IBF function.

Table 2 shows the Spearman's ranked correlation coefficient for the default algorithm generation pipeline, the pipelines that included different combinations of experimental parameters, inclusion of the IBF features, and other changes above improved the overall accuracy of prediction of IMWL by over 90%.

TABLE 2

| Test | Default | 1 | 2 | 3 | 4 | 1-3 | 1-4 |
|---|---|---|---|---|---|---|---|
| MLI | R | R | R | R | R | R | R |
| VAK | 0.69* | 0.82** | 0.69* | 0.71* | 0.69* | 0.86** | 0.76* |
| VAP | 0.60 | 0.64 | 0.60 | 0.62 | 0.27 | 0.73* | 0.59 |
| VDM | 0.41 | 0.81** | 0.41 | 0.17 | 0.29 | 0.52 | 0.79** |
| VHVS | 0.58 | 0.84** | 0.58 | 0.58 | 0.52 | 0.84** | 0.84** |
| VJH | −0.38 | 0.40 | −0.38 | −0.31 | −0.58 | 0.40 | 0.60 |
| VLP | 0.57 | 0.81** | 0.57 | 0.23 | 0.87** | 0.89** | 0.83** |
| VMC | 0.93** | 0.86* | 0.93** | 0.93** | 0.61 | 0.86* | 0.75 |
| VME | 0.47 | 0.49 | 0.47 | 0.22 | 0.60 | 0.74* | 0.84** |
| VMK | 0.69 | 0.73* | 0.69 | 0.69 | 0.68 | 0.73* | 0.40 |
| VMM | 0.96** | 0.94** | 0.96** | 0.83** | 0.94** | 0.94** | 0.91** |
| Average | 0.552 | 0.734 | 0.552 | 0.467 | 0.489 | 0.751 | 0.731 |

Table 2 shows that the 1-3 (eyes open and log₁₀ power of EEG band features) combination is the best when used with IBF, but the 1-4 combination (eyes open tasks and relative power) without IBF obtained the highest mean fit out of all combinations tested, and had an accuracy of 0.761.

Training the ML model for method 2 includes, for each patient, creating a training data frame from the patient's files containing EEG data for an activity or task performed by the patient. Each file is split into epochs of 800 samples with 600 sample overlap, and features values are calculated for each epoch. Epochs are windowed into 200 sample windows with 177 sample overlap. Since feature selection requires clean EEG data, and features from all electrodes are used, it is desirable to maximize the amount of clean data extraction from the training set. The large overlap is used to “scan” for 200 sample segments where all 4 electrodes have clean data. An overlap of 177 epochs is chosen such that window length—overlap (stride) is a prime number, ensuring that overlapping epochs contained unique windows. Feature values are then averaged across windows to achieve a single value for each feature, for every epoch.

A linear regression model is then trained on the feature values using the input from the patient after each activity/task using the NASA TLX scores as the dependent variable. Features are selected using a decision tree regressor, and features with importance greater than 0 are selected for use in the ML model. If more than 6 features have importance greater than 0, only the top 6 features are used. Several features could be used, including the ones discussed above and presented in Table 3 below:

TABLE 3

| Feature name | Description |
|---|---|
| Delta power | Standard frequency range for all users. 1-4 Hz evaluated on each |
| | electrode (4 features total). |
| Theta band power | Customized frequency range for each user between the end of the delta |
| | band and the beginning of the alpha band, determined on each |
| | electrode. Evaluated on each electrode (4 features total). |
| Low theta power | Standard frequency range for all users determined on each electrode. |
| | 4-6 Hz evaluated on each electrode (4 features total). |
| High theta power | Standard frequency range for all users determined on each electrode. |
| | 6-10 Hz evaluated on each electrode (4 features total). |
| Alpha band power | Customized frequency range for each user determined on each |
| | electrode. The peak alpha frequency (PAF) during rest is found. The |
| | low end of the alpha band is 2 Hz below the peak and the high end is 3 |
| | Hz above the peak. Evaluated on each electrode (4 features total). |
| Low alpha band | Customized frequency range for each user determined on each electrode. |
| power | PAF − 4 Hz to PAF − 2 Hz. Evaluated on each electrode (4 features total). |
| Mid alpha band | Customized frequency range for each user determined on each electrode. |
| power | PAF − 2 Hz to PAF evaluated on each electrode (4 features total). |
| High alpha band | Customized frequency range for each user determined on each electrode. |
| power | PAF to PAF + 2 Hz evaluated on each electrode (4 features total). |
| Low beta band | Customized frequency range for each user determined on each electrode. |
| power | PAF + 3 Hz to 16 Hz evaluated on each electrode (4 features total). |
| Very low beta band | Standard frequency range for all users. 13-15 Hz evaluated on each |
| power | electrode (4 features total). |
| Alpha area | The area within the polygon formed by joining PAF − 2 Hz and PAF + |
| | 3 Hz and the PSD values within that range. Evaluated on each electrode |
| | (4 features total). |
| Theta/alpha power | Standard frequency range for all users. 1-4 Hz/8-12 Hz evaluated on |
| ratio | each electrode (4 features total). |
| (Delta power + theta | Evaluated using the standard delta (1-4 Hz), theta (4-8 Hz) and alpha |
| power)/alpha power | (8-12 Hz) frequency bands evaluated on each electrode (4 features total). |
| Beta power 1 | Customized frequency range for each user determined on each |
| | electrode. The 6 Hz-wide band that is on average the highest in task |
| | compared to rest. Evaluated on each electrode (4 features total). |
| Beta power 2 | Customized frequency range for each user determined on each |
| | electrode. The 6 Hz-wide band that is on average the lowest in task |
| | compared to rest. Evaluated on each electrode (4 features total). |
| AF7/AF8 asymmetry | Evaluated using the customized theta, alpha, and low beta band ranges |
| in the theta band | and the standard 16-30 Hz high beta band range (4 features total). |
| Ln of high beta on AF7 − | Evaluated using the customized theta, alpha, and low beta band ranges |
| ln of high beta on AF8 | and the standard 16-30 Hz high beta band range (4 features total). |
| Ln of theta on AF7 − | Evaluated using the customized theta, alpha, and low beta band ranges |
| ln of theta on FPz | and the standard 16-30 Hz high beta band range (4 features total). |
| Ln of theta on AF8 − | Evaluated using the customized theta, alpha, and low beta band ranges |
| ln of theta on FPz | and the standard 16-30 Hz high beta band range (4 features total). |
| Spectral coherency | Evaluated using the customized theta, alpha, low alpha, mid alpha, high |
| between AF7 and | alpha, and low beta frequency bands and the standard high beta |
| AF8 | frequency bands (7 features total). |
| Imaginary spectral | Evaluated using the customized theta, alpha, low alpha, mid alpha, high |
| coherency between | alpha, and low beta frequency bands and the standard high beta |
| AF7 and AF8 | frequency bands (7 features total). |
| Phase locking value | Evaluated using the customized theta, alpha, low alpha, mid alpha, high |
| between AF7 and | alpha, and low beta frequency bands and the standard high beta |
| AF8 | frequency bands (7 features total). |
| Standard deviation | Evaluated on each electrode (4 features total). |
| Higuchi's fractal dimension | Evaluated on each electrode (4 features total). |
| Lempel-Ziv complexity | Evaluated on each electrode (4 features total). |

The ML model is then fitted to the feature values and TLX values from all multiplication tasks, all logic puzzle (visuospatial tasks), and the first eyes open rest recording in the engagement protocol. The instantaneous mental workload (IMWL) values are predicted for epochs from all tasks, and mean and standard deviation of IMWL values in multiplication and logic tasks are learned and stored for use in brain energy calculations. The mean and standard deviation of IMWL values in all eyes open rest tasks are learned and stored to assess performance of the ML model, and once a desired performance level is achieved, the ML model is considered trained and ready for use.

In some embodiments, epoch length and epoch overlap default values in the app 228 are set to 2211 and 201 samples, respectively. The app 228 accordingly receives data from the EEG headset 202 accordingly. Brain energy is calculated for each epoch of each task. Each epoch is windowed into windows of 200 samples with an overlap of 100 samples, and features identified in the training process were calculated for each window. An IMWL score is determined or predicted by the ML model from the feature values of each window. The IMWL scores for windows that are not contaminated by noise, blinks, eye motion, or packet loss are averaged together to obtain a single IMWL value for the epoch.

Brain energy used (BEU) is calculated for each epoch using the IMWL score and the parameters of the task distribution learned during training. In particular, a z-score is calculated for each IMWL score as:

$${z}_{task} = \frac{IMWL - task\_mean}{std\_task}$$

Calculating BEU for a single epoch i (BEU(i)) is calculated using the following formula, Large changes in brain energy used (BEU) for a epoch i are smoothed using a smoothing function calculated as:

$$BEU ( i ) = \frac{1}{{e}^{- ( 2 * Ztask ( i ) )} + 1}$$

The smoothing returns a value of BEU between 0 and 1 per epoch. The smoothing function was tested for a set of 6 patients, and was found to afford several advantages, such as creation of the best overall correlations between BEU for each task and task load index (TLX) scores for such tasks, generation of BEU slopes that are most consistent between 5-minute and 10-minute training tasks, and production of BEA that is most consistent between 5-minute and 10-minute training tasks.

Determining or calculating brain energy used (BEU) across a period of time of N epochs utilizes the relation:

BEU=Σᵢ₌₁^{N}BEU(i)

where:

BEU=Brain Energy used from 1 epochs to N epochs,

BEU(i)=Rate of Brain Energy used=Brain Energy used in one epoch i

i=epoch index

N=Total number of epochs

Therefore, a unit of brain energy used=amount of brain energy exerted in one epoch by patient while completing their most difficult activity/task. Brain energy used (BEU) by patient is calculated using the definition of one brain energy unit being equivalent to one epoch of maximal mental workload or cognitive exertion.

Training of the ML model to predict the IMWL in method 2 differs from the training of the ML model for method 1. In method 1, the labels used as dependent variable to learn the coefficients of the linear regression model of IMWL were the NASA TLX scores. The NASA TLX score used to capture the cognitive exertion induced during the Engagement Protocol was a percentage of the participants' actual maximum mental workload, and if a user self-report of NASA TLX were 0 for task 1, 25 for task 2 and 50 for task 3 during the engagement protocol, then the labels used for supervised machine learning were as stated by the patient 0, 25, and 50 for tasks 1, 2 and 3 respectively. In method 2, it is assumed that the engagement protocol induced maximal cognitive exertion in all participants and used the maximum reported TLX score in the training set. Accordingly, in method 2, the reported NASA TLX scores are normalized before being used as labels in the supervised machine learning of the ML model. For example, if a patient's NASA TLX scores were 0, 25, and 50, the method 2 infers that in each task the patient was working at 0%, 50%, and 100% of their maximum, and therefore, 0%, 50% and 100% were the labels used in supervised machine learning to predict IMWL in method 2.

Next, the brain energy used is calibrated by including scaling factors. The first step is to account for the rate at which the algorithms provide brain energy used values. The epoch length and epoch overlap specifications affect how many brain energy results are returned per second. Under the definition of one brain energy used unit, the possible brain energy used ranges from 0 to 1 unit per second. If the epoch parameters return brain energy used values at a different frequency than once per second, the brain energy used values must be scaled so that the definition of one brain energy used holds. In some embodiments, the app 228 is set to use an epoch length of 2211 samples (11 measurement frames) and an epoch overlap of 201 samples (1 measurement frame) to collect the EEG data via the EEG headset 202, from the patient 232. In some embodiments, the sampling rate of the EEG headset is set to 200 samples per second. Therefore, the number of results per second is:

$$rate = \frac{200 samples}{second} \times \frac{1 epoch}{2211 - 201 samples} \times \frac{1 result}{epoch} \\ rate = \frac{0.09950248756218905 results}{second}$$

A change in the epoch length and epoch overlap specifications influence on how quickly change in brain energy can occur. Epoch specification multiplier (ESM) is a scaling factor applied to the brain energy used result of each epoch to maintain the theoretical limits of one brain energy used nit per second. The ESM using the epoch specifications above is then:

$$ESM = \frac{1}{rate} \\ ESM = 10.05$$

Expected brain energy used (BEU) is the brain energy used based on a sustained mental workload of TLX_score_task summed across the total number of epochs. The expected BEU for a single EEG session or task is then estimated as:

$$BEU = total epochs \times ESM \times \frac{TLX score\_task}{TLX {score}_{max}}$$

The expected BEU is also related to the quality of the EEG data. For epochs that are completely contaminated by noise or packet loss, no BEU value is returned. For such epochs, it is impossible to estimate a Brain Energy value because the data is unusable. Therefore, to avoid overestimating expected BEU, signal quality and packet loss in the EEG data needs to be controlled. The change in BEU is adjusted by the proportion of epochs for which BEU values were returned.

$$BEU = total epochs \times ESM \times \frac{TLX score}{TLX {score}_{max}} \times \frac{total useable epochs}{total epochs}$$

Next, BEU is scaled such that amount of predicted BEU during the multiplication and logic tasks matches the expected BEU. The rest conditions are excluded from the scaling calculation because TLX scores are not assigned for such tasks. Only activities/tasks for which the patient provided an assessment of the mental workload (e.g., a TLX score) were used for determining how the BEU should be scaled.

BEU (predicted) is found by summing across all epochs for all tasks in the Engagement Protocol (excluding periods of rest eyes open and eyes closed) using the equation for rate as above, and the Brain Energy scaling factor (BESF) is calculated as:

$$BESF = \frac{{BEU}_{expected}}{{BEU}_{predicted}}$$

The change in BEU for each epoch then becomes

BEU_{epoch}=BEU_{predicted}×ESM×BESF

BEU of BESF scales the BEU to represent one second of maximal mental workload or cognitive exertion. For example, one minute of patient task where the patient used their highest cognitive exertion was 30, however, it is scaled up to 60 so that the units are one second of maximal cognitive exertion.

The amount of BEU depends on the rate at which results are given to the patient and the BESF. To simplify the calculation of BEU, these factors are combined into a single multiplier, called the Brain Energy multiplier (BEM). The BEM is calculated as:

BEM=ESM×BESF

and BEU for an epoch is calculated as:

BEU_{epoch}=BEU_{predicted}×BEM

For example, beginning of the day or a session, a patient starts with BEI of 3600. Brain energy available (BEA) at epoch N is the brain energy left after a set of activities during the course of the day is calculated by summing BEU per epoch across all N epochs and subtracting from BEI as:

BEA(N)=3600−Σᵢ₌₁^{N}BEU(i)

The sigmoid q parameter adjusts the steepness of the activation function, and lower values of q increase the function's output for z-scores <0 and decrease the function's output for z-scores >0. The function always returns a value of 0.5 when the z-score is 0, no matter what value q is. It was found that adjusting q for the entire function did not reliably yield expected Brain Energy values. Instead, the activation function was made discontinuous, with one q value for z-scores <0 and another for z-scores >=0. Values of q from 0.05 to 4 in increments of 0.05 were tested independently for z-scores <0 and z-scores >=0. Using a grid search, the combination of q values that was the closest match to the expected Brain Energy was selected for each user.

In some embodiments, a method 3 is used to determine BEU from the IMWL predicted by the ML model. The method 3 shares most of the same steps with method 2 as discussed above, except how the Brain Energy Scaling Factor (BESF) is calculated. It is theorized that the severity and location of a concussion can affect the extent to which a patient can exert themselves cognitively. For example, two individuals performing the same task at the same perceived level of cognitive exertion may experience different rates of cognitive fatigue. Therefore, it is possible that converting IMWL scores to BEU values using a single function may overestimate or underestimate the amount of brain energy used for some individuals. This is resolved by scaling up the BEU values.

For example, analogous to the treadmill test, where patients run on a treadmill until symptoms occur to learn their maximum heart rate for safe physical exertion, patients perform on-boarding tasks until they experience symptoms, or until 60 minutes have elapsed, whichever happens first. If the participant experiences symptoms, the total predicted BE up until symptoms occurred is the un-normalized BEU needed to define where their BBA occurs. If the patient does not experience symptoms, then they are asked to estimate how much longer they could continue the tasks until symptoms were triggered. In case of the BEU, the un-normalized BEU is extrapolated from the total predicted BEU during the 60 minutes to the time point estimated by the patient. The amount of BEU predicted for each epoch is then scaled up to the standard value for all used for all patients. In this manner, each patient's BEU are customized, but are displayed on the same scale as all patients. Brain Break Alert Threshold (BBAT) is calculated as:

Brain Break Alert Threshold (BBAT)=BEI−(BEU used to reach BBA)

The above definition is usable for determining the BBAT in other methods as well. Further,

BESF=(BEU used to reach BBA)/(sum of un-normalized predicted BEU across all epochs until symptom occurs)

where BESF=Brain Energy Scaling Factor, which is a scaling factor unique to each patient used to normalize their BEUs into standard units. (BEU used to reach BBA) is the standard value of BEU we are trying to normalize towards. (sum of predicted BEU across all epochs until symptom occurs) is the equation BEU=Σᵢ₌₁^{N}BEU(i) where BEU(i) are un-normalized values of Brain Energy Used (BEU).

Further, predicted BEU per epoch is given by:

$$BEU ( predicted ) = \frac{1}{{e}^{- ( 2 * {Z}_{task} )} + 1}$$

and

BEU(epoch)=BESF*(predicted un-normalized BEU per epoch i).

The BEU(i) calculated according to method 3 is then used for calculating BEA as:

$$BEA ( N ) = 3600 - {\sum }_{i = 1}^{N} BEU ( i )$$

In some embodiments, training the ML model for method 3 for data acquired during the Engagement Protocol include determining the number of epochs during the Engagement Protocol that elapsed before the patient experienced a symptom. The number of epochs is called s. Further, the predicted IMWL per each of the s epochs is determined using equation IMWL=b1*EEGf1+b2*EEGf2+ . . . +bn*EEGfn+c, and next, the mean of predicted IMWL across epochs for all non-rest activities in the Engagement Protocol is determined, and referred to as task_mean. Further, the standard deviation of predicted IMWL across epochs for all tasks (i.e. non-rest activities) in the Engagement Protocol is determined, and referred to as std_task. For each of the s epochs, the Z-score per each of the s epochs in the Engagement Protocol is determined by:

$${Z}_{task} = \frac{IMWL - task\_mean}{std\_task}$$

For each of the s epochs, calculate the un-normalized predicted BEU in the Engagement Protocol by the equation:

$$BEUU ( i ) = \frac{1}{{e}^{- ( 2 * Ztask ( i ) )} + 1}$$

where BEUU is the un-normalized predicted BEU. Across all of the s epochs, a sum of the un-normalized predicted BEU in the Engagement Protocol is determined using the equation:

BEUUS=Σᵢ₌₁^{S}BEUU(i)

where BEUUS is the sum of un-normalized predicted BEU across all epochs until symptom occurs.

In the present example, we select 300 Brain Energy Units as the standard value towards which scaling is done. BESF is given by BESF=300/(sum of un-normalized predicted BEU across all epochs until symptom occurs), that is, BESF=300/BEUUS.

In this manner, all the models and parameters needed to determine BEA, which is used to determine Brain Break Alerts are trained, and are stored in the BBA module 224. In some embodiments, the parameters include, coefficients to determine IMWL: b1, b2, . . . bn, c; task_mean of IMWL distribution; std_task of IMWL distribution; BEU used to reach BBA is arbitrarily set as 300 as the standard in this example, and 300 would be used for all patients as we are trying to standardize each patient BEU to reach BBA at 300; BESF; and BEI set as 3600 in this example.

Next, the trained model is used to determine the BEA and if Brain Break Alert has occurred are performed, and begin with determining the predicted IMWL per each of epoch using equation

IMWL=b1*EEGf1+b2*EEGf2+ . . . +bn*EEGfn+c,

determining the Z-score per each of epoch using the equation

$${Z}_{task} = \frac{IMWL - task\_mean}{std\_task} ,$$

determining the un-normalized BEU per epoch as:

$$BEUU ( i ) = \frac{1}{{e}^{- ( 2 * Ztask ( i )} ) + 1} .$$

Next, the normalized BEU is determined for each epoch by using equation

BEU(i)=BESF*BEUU(i),

and the BEA is determined up until the present moment (say after N epochs have elapsed) using the equation

BEA(N)=3600−Σᵢ₌₁^{N}BEU(i).

If BEA(N) is less than BBAT, a Brain Break Alert is generated.

The algorithm in method 3 may be enhanced further by training with additional training data acquired as the patient uses the EEG headset according to this method 3. For example, the patient can be asked at the end of each activity what their level of mental workload was, which is used to provide additional training data in addition to training data acquired during the Engagement Protocol. The additional training data is usable to refine the ML model for accuracy and/or adjust the model as the patient's brain changes. In some embodiments, method 3 additionally includes adjustments for lost epochs and different sizes of epochs, as disclosed with respect to method 2.

In some embodiments, methods disclosed herein are customizable according to the patient. In some embodiments however, the same methods can be used to create a general method that uses fixed EEG bands and fixed EEG features to create a one-size fits all model suitable for all users. In some embodiments, the methods include a hybrid approach of creating a semi-customized model, in which some features and EEG bands are fixed while keeping other features customized according to patient.

As discussed above the BEA is tracked over time and when the BBA becomes equal to or lower than a BBA threshold, a BBA is generated to notify the patient that the patient should take a break from the activity that the patient is engaged with. Further, recovery or return to work or to play requires a gradual increase in cognitive activity from the patient 232. Immediately after a concussion, a patient needs to start at low levels of cognitive exertion, and as and when the patient is able to exert their brain without triggering symptoms then they may increase the level at which they can work their brain, i.e., the total amount of Brain Energy they may exert in a given day. This gradual increase continues for a number of levels until the patient has healed and is able to return to their work and or sport at a similar level before they experienced a concussion. This level is known as the Brain Break Alert Difficulty Level, which is adjusted by changing the threshold at which the Brain Break Alert occurs. The BEA is used to determine when a Brain Break Alert occurs. In some embodiments, the brain break alert difficulty levels have 5 levels of difficulty. Difficulty Level 1 is conservative as a low amount of cognitive exertion is allowed before the patient receives a Brain Break Alert, while Difficulty Level 5 allows for the greatest amount of cognitive exertion before the brain break alert is generated.

BBAT is set using the number of seconds of maximal cognitive exertion. In this example, patients start the day at BEA of 3600. After 5 minutes of maximal cognitive exertion the patient depletes their Brain Energy by 300 leaving a BEA of 3300 (3600−5*60=3300). Using 300 is an example, other BBAT levels may be used. At Difficulty Level 1, a Brain Break Alert occurs at this point. After taking the recommended Brain Break (e.g. recommended by physician or a good night's sleep), the BEA is reset, allowing for another 5 minutes of maximal cognitive exertion before the next Brain Break Alert. When the patient has no symptoms at Level 1 and or they find it easy, their physician may decide to increase their cognitive exertion and raise the patient to difficulty level 2. The next day the patient starts at 3600 after waking up but since they are at level 2 they are allowed 10 minutes of maximal cognitive exertion until their Brain Break Alert occurs at BEA of 3000 (3600−10*60=3000). The patient progresses over to higher difficulty levels in a similar way. The BBA threshold can also be modified based on patients reporting symptoms prior to the Brain Break Alert. Also, as the patient shows signs of clinical improvement, their BBA threshold is adjusted to reflect their increased capacity for additional cognitive exertion.

FIG. 11A illustrates a front view of an EEG headset 202 worn by a patient, for example, the patient 232, in accordance with some embodiments. The EEG headset 202 includes a frame or a substrate 1102, and electrodes corresponding to desired contact points Fpz 1104, Fp1 1106, AF8 1108 and AF7 1110 on the forehead of the patient 232 and electrodes that contact the left and the right ears along the EEG headset 202, in order to contact the head of the patient 232. According to some embodiments, Fpz is placed at about 10% of distance from naison to inion, each of Fpz to Fp1, and Fp1 to AF7 is about 10% of half circumference spanning the cross-section of the head at the forehead level, and AF8 is about 20% of the half circumference spanning the cross-sections of the head at the forehead level. In some embodiments, electrodes are made using conductive fabric using silver thread or plastic coated with silver or silver chloride.

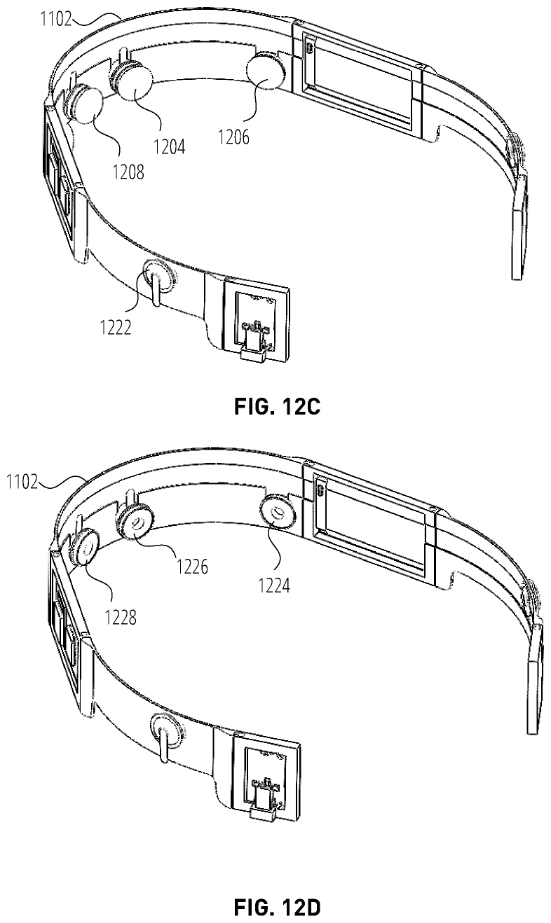

While only 4 contact points are visible in the illustration of FIGS. 11A and 11B, the EEG headset 202 may include electrodes corresponding to a fewer or a higher number of contact points, for example, contact points at or behind the top of the ears, for example, as shown in FIG. 12B. Such an inclusion of electrodes behinds the ears would amount to a total of 6 contact points, however, in some embodiments, the number of contact points may be higher or lower.

FIG. 11B illustrates a rear view of the EEG headset 202 of FIG. 11A, in accordance with some embodiments. The EEG headset 202 includes a biasing connector 1112 extending from either sides of the substrate 1102, and configured to reduce or tighten the diameter of the EEG headset 202 around the head of the patient 232 to tighten the EEG headset 202 to help with a snug fit and good contact of the electrodes and the head of the patient 232. The contact points are dimensioned according to the known small and large head sizes, and to accommodate the reduction in diameter due to tightening by the biasing connector 1112. Several mechanisms known in the art may be used for the biasing connector 1112.

FIG. 12A illustrates a perspective view of an EEG headset 202 without a biasing connector, in accordance with some embodiments. The EEG headset 202 includes an outer skin 1202 covering the inside (for facing the head of the patient 232) and the outside (opposite the inside) of the EEG headset 202. The outer skin 1202 is made from a material that is comfortable on human skin, such as a fabric, for example, made from cotton, or other absorbent and flexible fabrics. In some embodiments, the outer skin 1202 forms a closed surface via a zipper or a hook and look fastening arrangement. Contact pads or electrodes 1204, 1206, 1208 corresponding to contact points 1104, 1106, 1108, respectively, emerge from the outer skin 1202, and are configured to contact the contact points 1104, 1106, 1108 on the head of the patient 232. The electrode corresponding to the contact point 1110 is not visible in the views of FIGS. 12A-12D, however, exists in a manner similar to the contact pads or electrodes 1204, 1206, 1208.

The EEG headset 202 comprises an ear electrode region 1210 along the longitudinal substrate 1102 to accommodate both ears portion of the head of the patient 232. The ear electrode region 1210 may be worn over the ear or behind the ear, touching the head, and includes an ear electrode contact pad 1222 to contact a contact point on the ear or behind the ear through a conductive fabric or other type of electrode on the head of the patient 232. In some embodiments, the ear electrode region 1210 includes a recess to span around and accommodate the ear snugly, helping with keeping the EEG headset 202 stable on the head of the patient 232, and preventing a vertically downward motion or a rotational motion around the head of the patient 232. The electrode region 1210 as well as 1104, 1106, 1108, 1110 can be made from conductive fabric (e.g. using silver threads woven into fabric).

The EEG headset 202 also comprises a compartment for battery and other electronics 1212 and a processor and memory 1214. The compartment for battery and other electronics 1212 include a battery to power the electrodes, the processor and memory 1214, and other electronics, such as communication electronics, for example, as BLUETOOTH®, WIFI® or other wired or wireless communication electronics as known in the art. Other electronics may include one or more of a vibrating device (such as those found in smart phones), configured to vibrate the EEG headset 202, a speaker configured to generate a sound from the EEG headset 202, a display screen physically and/or communicably coupled to the EEG headset 202, among other suitable devices well known in the art, to alert the patient 232 wearing the EEG headset 202 of a notification or an alert, such as the brain break alert (BBA). The processor and memory 1214 are configured to pre-process the data captured by the electrodes (EEG data), for example, convert the electrical signals to digital signals, and/or send the EEG data with or without pre-processing to the user device 206 or to the system server 210, for example, via the communication electronics.

The electronics also include analog front end amplifiers that amplify the weak EEG signal and are electronics known in the art, an Analog to Digital Convertor to convert the analog EEG signals into digital (e.g. 24 bits per EEG sample), an impedance monitor to check for conductivity of the electrode contacts to the patient's skin, an antenna to communicate wirelessly, different types of memory that include flash memory to hold firmware code, RAM memory, a battery management system that controls charging and fuel gauge, user LED, Battery LED, power button, Bluetooth button, cabling to the electrodes (can be flex cable or individual wires, a circuit that provides driven ground (also known as driven right leg in the art), the electronics can also include other sensors such as accelerometers, gyroscopes, magnetometers, protection circuits to prevent electrocution of the patient when unit is charging, protection circuits to prevent electrostatic discharge damage of electronics, general purpose input/output pins that can be used for impedance checks or supplying reference voltages and test or development interfaces through the processor. In addition, other sensors can be added that allow the acquisition of physiological markers that are not part of the brain such as: photo-detector, microphones, optical based eye-tracking, Electrooculography based eye-tracking, heart rate variability and inter-beat intervals using electrocardiogram or pulse oximetry methods, electromyographic sensors to detect electrical activities of muscles.

FIG. 12B illustrates the EEG headset 202 of FIG. 12A without the outer skin 1202, in accordance with some embodiments. Removal of the outer skin 1202 exposes the longitudinal substrate 1102, the first cushioning element 1220 on the inside of the longitudinal substrate 1102 and the second cushioning element 1218 on the outside of the longitudinal substrate 1102. In some embodiments, the first cushioning element 1220 and the second cushioning element 1218 are foam material, with density and texture suitable to provide comfort to the patient 232 wearing the EEG headset 202 so that the patient 232 does not feel any of the solid features of the EEG headset 202. In some embodiments, the first cushioning element 1220 has a thicker profile along the front of the EEG headset 202 configured to engage with the forehead of the patient 232, and the first cushioning element 1220 thickness tapers down towards either sides of the head of the patient 232. The ear electrode region 1210 includes an ear electrode receptacle 1222, similar to the electrode receptacles on the forehead, and configured to capture EEG data from the head behind the ear, or from the ear. The non-conductive fabric of the headband can be constructed using for example combinations of nylon, polyester, elastane designed to be breathable and comfortable for long periods of time. Saline solution or other conductive electrolytes can be added to the conductive fabric at the time the headband is used to reduce impedance of the skin to electrode contact and therefore improve signal quality.

FIG. 12C illustrates the EEG headset 202 of FIG. 12A without the outer skin 1202 and cushioning elements 1220 and 1218, in accordance with some embodiments. Removal of the cushioning elements 1220 and 1218 exposes the longitudinal substrate 1102 and the electrodes 1204, 1206, 1208. The electrodes are conductive pads 1204, 1206, 1208 made from conductive material such as a conductive fabric or other conductive matrix, which is flexible and soft. The electrodes may be bonded to a metal button separated by cushioning material for added comfort. The electrodes are detachably attached to the longitudinal substrate 1102, for example, using a click fit mechanism, or other mechanisms as known in the art. The electrodes may also be attached directly to the substrate or sewn into non-conductive fabric skin or combinations thereof. For instance, the forehead electrodes may be detached but the ear conductive fabric electrodes are sewn into the non-conductive fabric of the outer skin.

FIG. 12D illustrates the EEG headset 202 of FIG. 12A without the outer skin 1202, cushioning elements 1220 or 1218, and conductive pads 1204, 1206, 1208, in accordance with some embodiments, which exposes the longitudinal substrate 1102 with the receptacles 1224, 1226 and 1228 for the conductive pads or electrodes 1204, 1206, 1208. The receptacles are configured to receive the conductive pads and attach the conductive pads removably to the longitudinal substrate 1102. Further, circuitry (not shown) electrically couples the electrodes, the processor and memory 1214 and the battery and other electronics. The receptacles corresponding to the ear electrode is shown by numeral 1222, and operates similar to the receptacles 1224, 1226 and 1228. The electrode 1222 may have any suitable shape, such as circular, rectangular, irregular shape or a combination thereof. Other electrodes similar to such electrodes may be included and operate in a similar manner to such electrodes.

In some embodiments, the longitudinal substrate 1102 is a thin, flexible band that can be laid in a flat configuration, to enable convenient assembly of various components described with respect to FIGS. 12A-12D onto the longitudinal substrate 1102. For example, the longitudinal substrate 1102 includes a cavity compartment for battery and other electronics 1212, a cavity of the processor and memory 1214, ear electrode region 1210 and connectors 1216. Cushioning elements 1220 and 1218 are assembled onto the longitudinal substrate 1102, for example, using an adhesive or hook and loop fasteners. The first cushioning element 1220 includes holes for the electrodes/conductive pads to engage with the receptacles. Next, the outer skin 1202 is worn over the cushioning elements 1220 and 1218 to enclose the entire cushioning pads and a substantive portion of the longitudinal substrate 1102. The outer skin 1202 uses a zipper or a hook and loop fastener to enclose the components therein, and the outer skin 1202 includes holes corresponding to the cushioning elements for the conductive pads/electrodes. Next, conductive pads/electrodes are assembled to engage with the receptacles to complete the EEG headset 202. Once assembled, the longitudinal substrate 1102 assembled with components, including the biasing connector 1112, the EEG headset 202 can be worn in the configuration of a curve of the head of the patient 232.

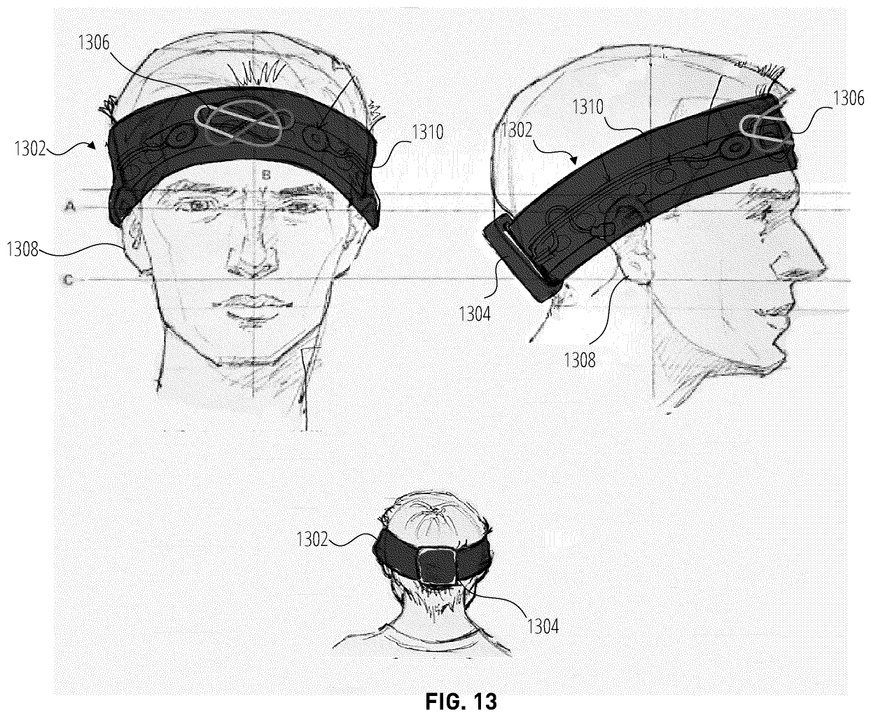

FIG. 13 illustrates several views of an EEG headset 1302, similar to the EEG headset 302, in accordance with some embodiments. The EEG headset 1302 is designed to hold a plurality of electroencephalogram (EEG) electrodes on the head and enable easy, prolonged, and comfortable measurement of EEG data. The electrodes are powered by a re-chargeable lithium battery and send data to a secured cloud storage system via Bluetooth or Bluetooth Low Energy.

The external material 1310 of the EEG headset 1302 allows for a comfortable fit over the forehead of the patient. The EEG headset 1302 can either be firm or made of material that stretches—so long as the EEG headset 1302 remains secured to the head. The external material 1310 can wick sweat; it can also attach to Velcro™. A plurality of electroencephalogram (EEG) electrodes are placed on the forehead and clipped to the ear 1308 to evaluate the electrical activity in the brain. In the embodiment shown in FIG. 13, a logo 1306 is shown on the external material 1310.

The external material 1310 is tight enough to hold a board casing 1304 sleeve within. The board casing 1304 fits comfortably into the small of the neck under the inion of the skull.

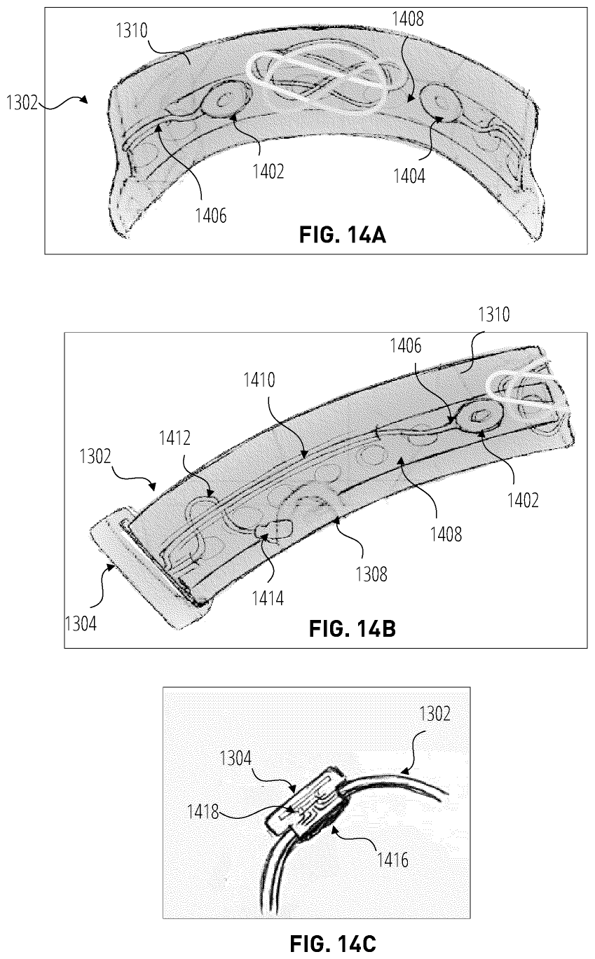

FIG. 14A illustrates several views of an EEG headset 1302 in accordance with some embodiments. In FIG. 14A, the external material 1310 of EEG headset 1302 has been rendered translucent to show an embodiment of electrode 1402 and electrode 1404 that are in contact with an patient's frontal lobe. Each electrode is attached to a sheet of elastic material 1408 that is secured to the underside of the external material 1310. The elastic material 1408 can be secured to the external material 1310 using means known in the art; an example includes a Velcro™ fabric board. EEG headset 1302 is designed for comfortable long-term wearability; surfaces of EEG headset 1302 that make contact with the skin can be padded to provide comfort. In some embodiments, EEG headset 1302 is padded with foam sewn into breathable clothing material.

In some embodiments, dry sensors (or electrodes) are used, thereby precluding the need for skin abrasion, which introduces the risk of infection and pain for the subject. The electrodes may also be spring mounted to help improve signal quality and comfort to the patient. In some embodiments, the electrode tips of the biosensors are coated with Ag/AgCl which is commonly used in FDA-approved EEG and ECG electrodes. In other embodiments the electrodes are biocompatible conductive silicone rubber.

FIG. 14B illustrates a side view of a sensor interface in accordance with some embodiments. Elastic material 1408 also includes a sleeve track 1410 for electrode wires; in FIG. 14A, wire 1406 of electrode 1402 is shown in sleeve track 1410. A similar arrangement exists on the other side of the elastic material 1408 for electrode 1404. There is a second electrode 1414 that is secured to the patient's ear 1308 via a clip 1412. A similar electrode (not shown) can be clipped to the patient's other ear.

In the embodiment shown in FIG. 14A and FIG. 14B, two electroencephalogram (EEG) electrodes are placed on the forehead and one electrode is clipped to each ear to evaluate the electrical activity in the brain. The electrodes can be conductive rubber electrophysiological sensors that measure bioelectric signals using an operational amplifier per electrode to buffer the signal from the body. In some embodiments, each of the four input channels are buffered by an AD8237 Instrumentation Amplifier with a differential input impedance of 100 MΩ. The EEG circuit board can be powered by a 3.7 V battery. The electrode material can be conductive rubber, and the interface between the skin and the electrode can be designed to ensure adequate contact with the skin without the use of an electrolytic gel.

Further details are as follows. The sensors are active, utilizing an operational amplifier to buffer the signal from the body. The biosensors can be optimized for low power operation, where the input amplifiers may operate from 3 V supply. The sensor input impedance may be greater than 100 MΩ at DC. This resistance appears in parallel with a capacitor less than 5 pF. Furthermore, Driven Ground is 1.5 V through a 330 KΩ resistor. Protection from electrostatic discharge may be provided using a Low Leakage ESD Protection Device—for example, a TPD4E1B06 4-Channel Ultra Low Leakage ESD Protection Device. Impedance measurements of connected electrodes can be measured by a signal generated by an AD5621 Digital Analog Convertor. Furthermore, the system can be powered entirely by a commercial lithium ion battery (NB-4L, 3.7V nom). In some embodiments Nickel-metal hydride batteries are used.

Several headband sizes can be created to fit a wide range of head sizes informed by the NASA anthropometry design considerations document. For a precise fit, electrodes within each headband may be attached to a thick fabric elastic band that allows the distance between electrodes to widen to the required distance depending on a patient's head circumference. This ensures that the data from each headset comes from the appropriate brain region, while allowing for a personalized adjustment. While four electrodes are shown in FIG. 14A and FIG. 14B, it is understood that fewer than four or more than four electrodes can be placed on the head of the patient, to acquire data. In some embodiments, three electrodes are attached to the patient's forehead (in addition to the two electrodes attached to the ears).

FIG. 14C illustrates cut-away side view of a board casing 1304 in accordance with some embodiments. Wires 1418 from the plurality of electrodes feed into board casing 1304, from their respective sleeve tracks in the EEG headset 1302. Board casing 1304 can have padding 1416 or fabric to serve as a cushion between the board casing 1304 and the patient's neck.

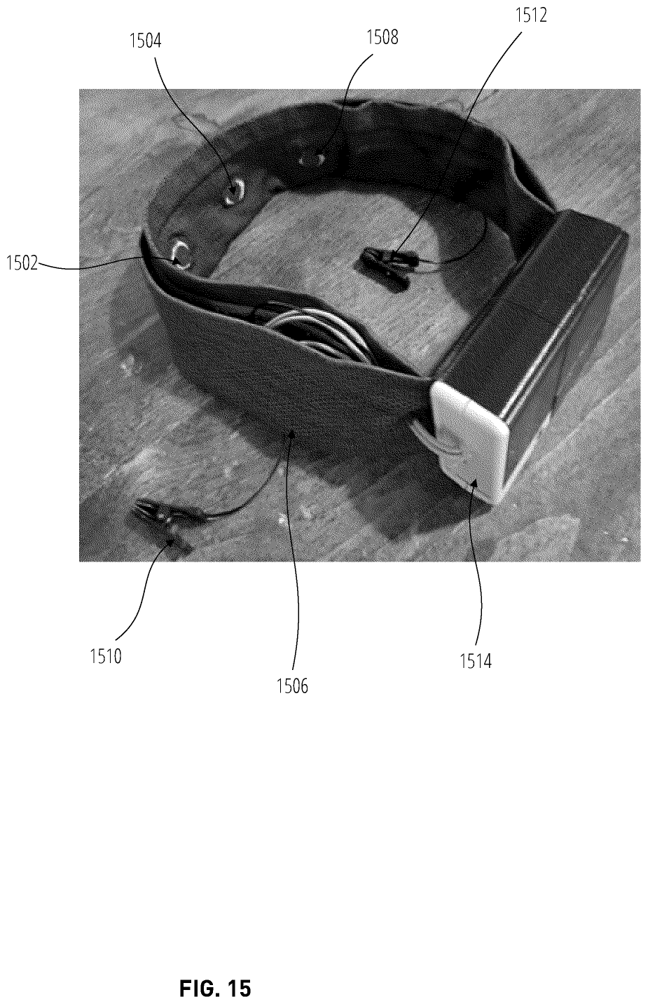

FIG. 15 illustrates a perspective view of an EEG headset 1506 in accordance with some embodiments. The EEG headset 1506 holds the electrodes 1502, 1504 and 1508 on the head and enable easy, prolonged, and comfortable electroencephalography measurement that can be streamed via Bluetooth to a privacy-compliant compliant, secure cloud. In the embodiment shown in FIG. 15, the EEG headset 1506 comprises three conductive electrodes 1502, 1504 and 1508 for contact with a patient's forehead, and two ear clips 1510 and 1512 for attachment to the patient's ears. Each of the ear clips contain electrodes and are part of the EEG system. Conductive electrodes 1502, 1504 and 1508 can be made of conductive silicone. The board casing 1514 fits at the back of the patient's head. In some embodiments, each ear clip 1510 and 1512 is replaced by an in-ear conductive silicone electrode, which fits into the patient's ear.

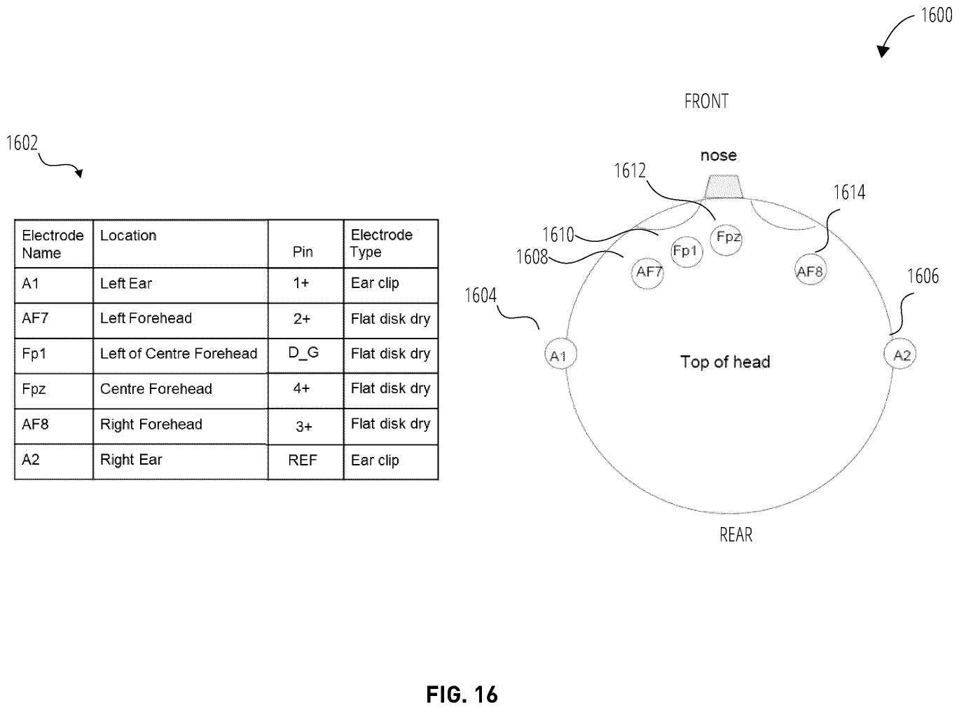

FIG. 16 illustrates an electrode montage 1600 in accordance with some embodiments, in which possible head locations for EEG electrodes are provided. The front, rear and top of the head are shown, along with the nose. In the montage 1600, six electrodes are shown: A1 (item 1604) at the left ear; A2 (item 1606) at the right ear; and four electrodes each at AF7 (item 1608), Fp1) item 1610), Fpz (item 1612) and AF8 (item 1614). The electrode at Fpz (item 1612) serves as a driven ground, which removes environmental noise from the signal. The remaining electrodes detect brain activity. Accompanying table 1602, summarizes the electrode name and location shown in montage 1600.

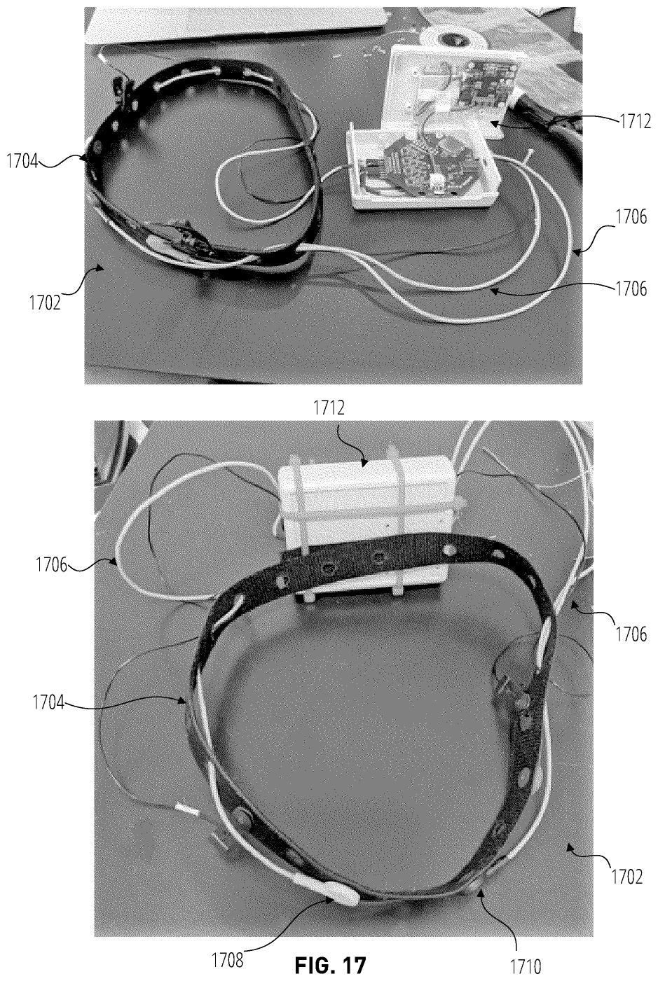

FIG. 17 illustrates partial assembly of an EEG headset 1702 in accordance with some embodiments. In FIG. 17, the EEG headset 1702 has been stripped of its external material, to show the sheet of elastic material 1704, wires 1706, electrodes 1708, 1710 and board casing 1712.

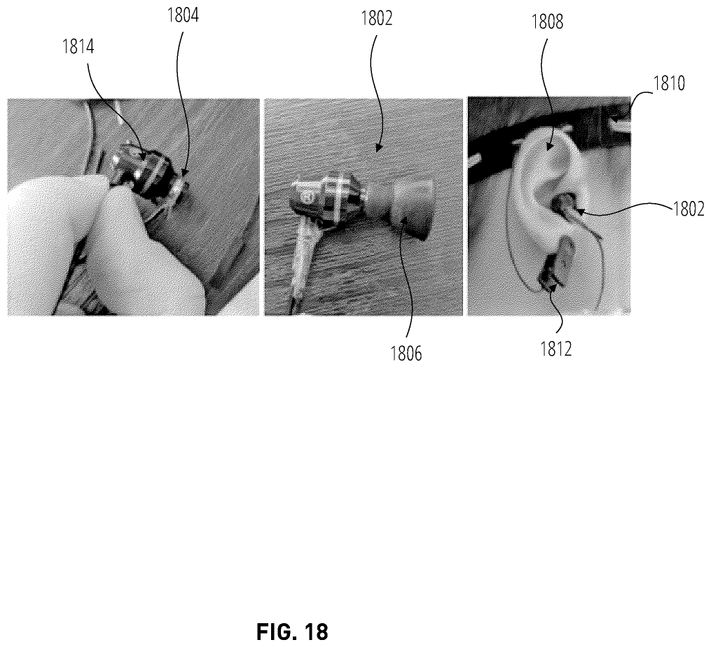

FIG. 18 illustrates an in-ear in ear electrode 1802 in accordance with some embodiments. The in ear electrode 1802 comprises an ear piece 1814 which is attached to an electrode 1804, which in turn is covered by a cushion 1806. The assembled in ear electrode 1802 fits comfortably in the ear 1808 of a patient and measure electrical signals emitted from the patient's brain. FIG. 18 also shows an ear clip 1812. However, both the ear clip 1812 and the in ear electrode 1802 may not be used at the same time, and only one type of ear electrode is selected.

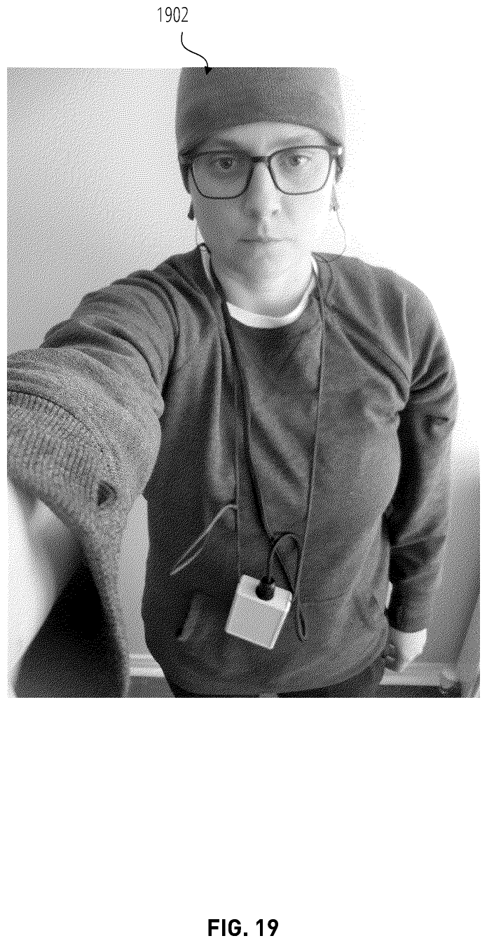

FIG. 19 illustrates EEG headset 1902 in accordance with some embodiments. In FIG. 19, EEG headset 1902 is in the form of a skullcap or tuque.

While EEG headset 1302 and EEG headset 1506 are illustrated, the systems and methods disclosed herein can use other EEG headsets as known in the art that convey data to a secure cloud-based data storage via Bluetooth. Since private medical data is being stored, the cloud-based data storage is compliant with all local privacy regulations and meets local research ethics board (REB) standards. For example, in the United States, the cloud-based data storage is compliant with the Health Insurance Portability and Accountability Act (HIPPA). The data collected from the patient is stored in the cloud-based data storage, and then sent to a machine-learning analytics engine. The patient's EEG history is tracked and provided to the patient to: alert him/her prior to an overload; and to share the information with the patient's team of physicians, clinicians, trainers and caregivers.

Various embodiments disclosed herein provide method and apparatus for concussion recovery. Embodiment 1 includes a method for generating a brain break alert comprises receiving, at a system server from a user device, electroencephalographic (EEG) data of a patient engaged in a cognitive activity, where the EEG data is captured by at least one electrode of an EEG headset, the at least one electrode is in physical contact with the patient, and the EEG headset is communicably coupled to the user device, determining, at the system server, brain energy used (BEU) by the patient based on the EEG data, determining, at the system server, brain energy available (BEA) to the patient as a function of the BEU and a predefined initial brain energy (BEI) of the patient, where the BEA is proportional to a difference between the BEI and the BEU, determining, at the system server, that the BEA has satisfied a brain break alert threshold, and sending, from the system server to the user device, upon the brain break alert threshold being satisfied, an instruction to generate a brain break alert (BBA) on the user device.

Embodiment 2 includes the method of embodiment 1, further comprising predicting, at the system server, based on the EEG data, an instantaneous mental workload (IMWL) of the patient, wherein the BEU is a cumulation of the IMWL.

Embodiment 3 includes the method of embodiment 2, wherein the IMWL is determined by a machine learning (ML) model, wherein the ML model is pre-trained using a training data of the patient.

Embodiment 4 includes the method of embodiment 3, wherein the ML model includes at least one of linear regression model, decision tree regression model, partial least squares model, another regression model, or a classifier model.

Embodiment 5 includes the method of embodiment 4, wherein the EEG data for an epoch is transformed to at least one feature, the at least one feature comprising a frequency domain feature, a time domain feature, a spatial feature, or a non-linear feature.

Embodiment 6 includes the method of embodiment 5, further comprising determining, at the system server, a feature parameter specific to the patient and the at least one electrode, wherein the at least one feature is the frequency domain feature.

Embodiment 7 includes the method of embodiment 6, wherein the ML model is a linear regression model, and the EEG data corresponds to a plurality of EEG features, wherein the linear regression model is defined as:

IMWL=b1*EEGf1+b2*EEGf2+ . . . bn*EEGfn+c,

where:

bn is the nth coefficient of the linear regression model,

EEGfn is a feature calculated from the EEG signal, and

c is a constant.

Embodiment 8 includes the method of embodiment 7, wherein the training data for the linear regression model includes a recording the EEG data for at least one training phase activity performed by the patient, and an input of the patient's assessment of their mental workload for the at least one training phase activity.

Embodiment 9 includes the method of embodiment 8, wherein the at least one training phase activity comprises at least one of rest, eyes open, eyes close, visuospatial tasks, arithmetic, logic puzzles, executive function, memorizing, or varying level of difficulty thereof.

Embodiment 10 includes the method of embodiment 2, wherein the BEU is determined for a plurality of epochs in the duration the patient is engaged in the cognitive activity, wherein the BEA is calculated as:

BEA(k)=BEI−Σᵢ₌₁^{k}BEU_{epoch}(i),

where “i” is an index having a value of 1 at the start of the cognitive activity, and

“k” is the total number of epochs.

Embodiment 11 includes the method of embodiment 1, wherein the BBAT is customizable.

Embodiment 12 includes the method of embodiment 11, further comprising:

increasing, at the system server, the BBAT by at least one of the passage of time, reported progress of the patient, or by a medical care provider of the patient.

Embodiment 13 includes the method of embodiment 1, further comprising:

identifying, at the system server, noise in the EEG data by a classifier ML model; and

removing, at the system server, noise from the EEG data.

Embodiment 14 includes the method of embodiment 13, wherein the noise includes at least one of a biological noise of the patient, a noise due to movement of the patient, noise due to detachment of the electrodes from the skin of the patient, or an environmental noise.

Embodiment 15 includes the method of embodiment 14, wherein the biological noise comprises at least one of an eye movement, a blink, or muscle tension.

Embodiment 16 includes the method of embodiment 1, wherein the user device and the EEG headset are remote to the system server.

Embodiment 17 includes the method of embodiment 1, wherein the brain break alert is generated in real time.

Embodiment 18 includes the method of embodiment 1, wherein the brain break alert is generated based on EEG data aggregated over a predefined period of time.

Embodiment 19 includes the method of embodiment 1, wherein the BEU is determined for a plurality of epochs spanning the duration the patient is engaged in the cognitive activity, the method further comprising:
adjusting the BEA downward proportional to the BEU for the epochs the BEU is greater than a first threshold; and
adjusting the BEA upward proportional to the BEU for the epochs the BEU is lower than a second threshold.

Embodiment 20 includes a method for generating a brain break alert, the method includes receiving, at a system server (1210) from an electroencephalographic (EEG) headset (1202), EEG data of a patient engaged in a cognitive activity, where the EEG data is captured by at least one electrode of the EEG headset worn by the patient, the at least one electrode in physical contact with the patient, the EEG headset communicably coupled to a user device (1206), determining, at the system server, brain energy used (BEU) by the patient based on the EEG data, calculating, at the system server, brain energy available (BEA) to the patient as a function of the BEU and a predefined initial brain energy (BEI) of the patient, where the BEA is proportional to a difference between the BEI and the BEU, determining, at the system server, that the ABE has satisfied a brain break alert threshold (BBAT), and sending, from the system server, to the user device or to the EEG headset, upon the BBAT being satisfied, an instruction to generate a brain break alert (BBA) on the user device or the EEG headset.

Embodiment 21 includes a computing apparatus including a processor and a memory storing instructions that, when executed by the processor, configure the apparatus to perform method(s) according to one or more of the embodiments 1-19.

Embodiment 22 includes a non-transitory computer-readable storage medium including instructions that when executed by a computer, cause the computer to perform method(s) according to one or more of the embodiments 1-19.

Embodiment 23 includes, in one aspect, an electroencephalographic (EEG) headset for being worn on a head of a patient, the EEG headset includes a longitudinal substrate includes an inner side configured to face the head of the patient when the EEG headset is worn on the head by the patient, an outer side opposite the inner side, a front configured to face the forehead when the EEG headset is worn on the head by the patient, sides configured to face the side of the head, respectively, when the EEG headset is worn on the head by the patient, a notch on each of the sides shaped to accommodate ears, respectively, when the EEG headset is worn on the head by the patient, a biasing connector extending from the substrate, the biasing connector configurable to reduce a combined circumference of the substrate and the biasing connector, causing the substrate to conform to the head of the patient when the EEG headset is worn on the head by the patient, at least one electrode corresponding to the at least one receptacle, the at least one electrode extending from the inner side, facing the head of the patient when the EEG headset is worn on the head by the patient, a first cushioning element disposed on the inner side of the substrate, the cushioning element having a thickness that tapers down from the front to the sides, a computing device and a memory communicably coupled to the computing device, a power source coupled to the computing device and to the at least one electrode.

Embodiment 24 includes the EEG headset of embodiment 23, and may also include where the at least one electrode is positioned to contact with at least one of above the ear, the center of the forehead, the left forehead, between the center of the forehead and the left forehead, or the right forehead.

Embodiment 25 includes the EEG headset of embodiment 24, and may also include where the at least one electrode includes a conductive fabric configured to contact the patient.

Embodiment 26 includes the EEG headset of embodiment 25, and may also include where the substrate further includes at least one receptacle corresponding to and configured to receive the at least one electrode, where the at least one electrode is attached removably to the substrate at the at least one receptacle.

Embodiment 27 includes the EEG headset of embodiment 26, and may also include further includes a second cushioning element on the outer side of the substrate covering the substrate uniformly.

Embodiment 28 includes the EEG headset of embodiment 27, and may also include further includes an outer skin configured to envelope the substrate, the first and the second cushioning elements, the outer skin having an opening to allow the at least one electrode to stay exposed.

Embodiment 29 includes the EEG headset of embodiment 28, and may also include where the longitudinal substrate is flexible.

Embodiment 30 includes, in one aspect, a method for generating a brain break alert, the method includes receiving, at a user device, from an electroencephalographic (EEG) headset communicably coupled to the user device, EEG data of a patient engaged in at least one training phase activity, the EEG data captured by at least one electrode of the EEG headset, the at least one electrode in physical contact with the patient, receiving, at the user device, an input of the patient's assessment of their mental workload for the at least one training phase activity, and sending, from the user device to a system server, the EEG data and the input of the patient's assessment of their mental workload.

Embodiment 31 includes the method of embodiment 30, and may also include where the at least one training phase activity includes at least one of rest, eyes open, eyes close, visuospatial tasks, arithmetic, logic puzzles, executive function, memorizing, or varying level of difficulty thereof.

Embodiment 32 includes the method of embodiment 31, and may also include further includes receiving, at the user device, from the system server, a description of the at least one training activity.

Embodiment 33 includes the method of embodiment 32, and may also include further includes receiving, at the user device, from the system server, a sequence of a plurality of training activities, where the at least one training activity includes the plurality of training activities.

Embodiment 34 includes the method of embodiment 33, and may also include further includes receiving, at the user device, from the EEG headset, EEG data for the patient engaged in a cognitive activity, sending, from the user device, to the system server, the EEG data, receiving, at the user device, from the system server, an instruction to generate a brain break alert (BBA), and generating, at the user device, the BBA.

One or more aspects of various embodiments described herein may be compatible with aspects of other embodiments, and all such permissible combinations are contemplated herein.

Various techniques described herein may be modified, as needed, using techniques known in the art, to comply with relevant regulations, such as Health Insurance Portability and Accountability Act (HIPAA) guidelines of the US or equivalent, patient privacy guidelines, and others. While one or more ML models are discussed herein, the methods are not restricted to the disclosed ML models, which are presented for reference purposes only. Several other ML models as known in the art may be suitable for being used with the techniques disclosed herein, and are contemplated herein. Moreover, aspects of certain embodiments may be compatible with aspects of certain other embodiments, and a combination of different aspects to arrive at embodiments not specifically described herein would readily occur to those skilled in art, and all such embodiments are contemplated herein.

A computer program (which may also be referred to or described as a software application, code, a program, a script, software, a module or a software module) can be written in any form of programming language. This includes compiled or interpreted languages, or declarative or procedural languages. A computer program can be deployed in many forms, including as a module, a subroutine, a stand-alone program, a component, or other unit suitable for use in a computing environment. A computer program can be deployed to be executed on one computer or can be deployed on multiple computers that are located at one site or distributed across multiple sites and interconnected by a communication network.

As used herein, a “software engine” or an “engine,” refers to a software implemented system that provides an output that is different from the input. An engine can be an encoded block of functionality, such as a platform, a library, an object or a software development kit (“SDK”). Each engine can be implemented on any type of computing device that includes one or more processors and computer readable media. Furthermore, two or more of the engines may be implemented on the same computing device, or on different computing devices. Non-limiting examples of a computing device include tablet computers, servers, laptop or desktop computers, music players, mobile phones, e-book readers, notebook computers, PDAs, smart phones, or other stationary or portable devices.

The processes and logic flows described herein can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit). For example, the processes and logic flows can be performed by and apparatus can also be implemented as a graphics processing unit (GPU).

Computers suitable for the execution of a computer program include, by way of example, general or special purpose microprocessors or both, or any other kind of central processing unit. Generally, a central processing unit receives instructions and data from a read-only memory or a random access memory or both. A computer can also include, or be operatively coupled to receive data from, or transfer data to, or both, one or more mass storage devices for storing data, e.g., optical disks, magnetic, or magneto optical disks. It should be noted that a computer does not require these devices. Furthermore, a computer can be embedded in another device. Non-limiting examples of the latter include a game console, a mobile telephone a mobile audio player, a personal digital assistant (PDA), a video player, a Global Positioning System (GPS) receiver, or a portable storage device. A non-limiting example of a storage device include a universal serial bus (USB) flash drive.

Computer readable media suitable for storing computer program instructions and data include all forms of non-volatile memory, media and memory devices; non-limiting examples include magneto optical disks; semiconductor memory devices (e.g., EPROM, EEPROM, and flash memory devices); CD ROM disks; magnetic disks (e.g., internal hard disks or removable disks); and DVD-ROM disks. The processor and the memory can be supplemented by, or incorporated in, special purpose logic circuitry.

To provide for interaction with a patient, embodiments of the subject matter described herein can be implemented on a computer having a display device for displaying information to the patient and input devices by which the patient can provide input to the computer (e.g. a keyboard, a pointing device such as a mouse or a trackball, etc.). Other kinds of devices can be used to provide for interaction with a patient. Feedback provided to the patient can include sensory feedback (e.g. visual feedback, auditory feedback, or tactile feedback). Input from the patient can be received in any form, including acoustic, speech, or tactile input. Furthermore, there can be interaction between a patient and a computer by way of exchange of documents between the computer and a device used by the patient. As an example, a computer can send web pages to a web browser on a patient's client device in response to requests received from the web browser.

Embodiments of the subject matter described in this specification can be implemented in a computing system that includes: a front end component (e.g., a client computer having a graphical patient interface or a Web browser through which a patient can interact with an implementation of the subject matter described herein); or a middleware component (e.g., an application server); or a back end component (e.g. a data server); or any combination of one or more such back end, middleware, or front end components. The components of the system can be interconnected by any form or medium of digital data communication, e.g., a communication network. Non-limiting examples of communication networks include a local area network (“LAN”) and a wide area network (“WAN”).

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

The methods described herein may be implemented in software, hardware, or a combination thereof, in different embodiments. The methods may be embodied in computer instructions stored in a non-transitory computer readable medium, and executed by one or more processors to perform the methods. In addition, the order of methods may be changed, and various elements may be added, reordered, combined, omitted or otherwise modified. All examples described herein are presented in a non-limiting manner. Various modifications and changes may be made as would be obvious to a person skilled in the art having benefit of this disclosure. Realizations in accordance with embodiments have been described in the context of particular embodiments. These embodiments are meant to be illustrative and not limiting. Many variations, modifications, additions, and improvements are possible. Accordingly, plural instances may be provided for components described herein as a single instance. Boundaries between various components, operations, and data stores are somewhat arbitrary, and particular operations are illustrated in the context of specific illustrative configurations. Other allocations of functionality are envisioned and may fall within the scope of claims that follow. Finally, structures and functionality presented as discrete components in the example configurations may be implemented as a combined structure or component. These and other variations, modifications, additions, and improvements may fall within the scope of embodiments as defined in the claims that follow.

While the foregoing is directed to embodiments of the present invention, other and further embodiments of the invention may be devised without departing from the basic scope thereof, and the scope thereof is determined by the claims that follow.

1-15. (canceled)

16. A method for generating a brain break alert (BBA), the method comprising:
providing a system server connected to a network and configured to store at least one algorithm for concussion recovery;
securing, an electroencephalographic (EEG) headset to the head of a patient engaged in a cognitive activity;
transmitting, to the system server from a user device, EEG data of the patient engaged in a cognitive activity, wherein the EEG data is captured by at least one electrode of the EEG headset, the at least one electrode is in physical contact with the patient, and the EEG headset is communicably coupled to the user device;
determining, at the system server, that a brain energy available (BEA) to the patient has satisfied a brain break alert threshold (BBAT); and
receiving, at the user device from the system server, upon the brain break alert threshold being satisfied, an instruction to generate the BBA on the user device, wherein the BBA effects a change in cognitive activity done by the patient that helps the patient brain to rest and recover.

17. The method of claim 16, wherein the BEA to the patient is a function of a predefined initial brain energy (BEI) of the patient and a brain energy used (BEU) by the patient based on the EEG data.

18. The method of claim 16, wherein the BEA to the patient is proportional to a difference between a predefined initial brain energy (BEI) of the patient and a brain energy used (BEU) by the patient based on the EEG data.

19. The method of claim 16, wherein the change in cognitive activity done by the patient comprises a cognitive activity selected from a group consisting of meditation, sleep, rest in a peaceful environment, listen to relaxing music, guided stress reduction, guided heart rate biofeedback, guided neurofeedback biofeedback, relaxing hobby, gentle yoga, breathing exercises designed to help one relax, physical exercise, playing a game, having a conversation, walking a dog and any combination thereof.

20. A system for generating a brain break alert (BBA) comprising:
a system server connected to a network and configured to store at least one algorithm for concussion recovery;
an electroencephalographic (EEG) headset secured to the head of a patient engaged in a cognitive activity, wherein the EEG data of the patient engaged in a cognitive activity is captured by at least one electrode of the EEG headset, the at least one electrode is in physical contact with the patient;
a user device communicably coupled to the EEG headset;
wherein the system server determines that a brain energy available (BEA) to the patient has satisfied a brain break alert threshold (BBAT), transmits an instruction to generate the BBA to the user device and the BBA effects a change in cognitive activity done by the patient that helps the patient brain to rest and recover.

21. The system of claim 20, wherein the BEA to the patient is a function of a predefined initial brain energy (BEI) of the patient and a brain energy used (BEU) by the patient based on the EEG data.

22. The system of claim 20, wherein the BEA to the patient is proportional to a difference between a predefined initial brain energy (BEI) of the patient and a brain energy used (BEU) by the patient based on the EEG data.

23. The system of claim 20, wherein a change in cognitive activity done by the patient comprises a cognitive activity selected from a group consisting of meditation, sleep, rest in a peaceful environment, listen to relaxing music, guided stress reduction, guided heart rate biofeedback, guided neurofeedback biofeedback, relaxing hobby, gentle yoga, breathing exercises designed to help one relax, physical exercise, playing a game, having a conversation, walking a dog and any combination thereof.

24. A system for generating a brain break alert (BBA) comprising:
a system server connected to a network and configured to receive from a user device, electroencephalographic (EEG) data of a patient engaged in a cognitive activity;
EEG data of the patient engaged in a cognitive activity is captured by at least one electrode of an EEG headset, the at least one electrode is in physical contact with the patient, and the EEG headset is communicably coupled to the user device;
said system server is configured to store at least one algorithm for concussion recovery;
wherein the system server determines that brain energy available (BEA) to the patient has satisfied a brain break alert threshold (BBAT), transmits an instruction to generate the BBA to the user device and the BBA effects a change in cognitive activity done by the patient that helps the patient brain to rest and recover.

25. The system of claim 24, wherein the BEA to the patient is a function of a predefined initial brain energy (BEI) of the patient and a brain energy used (BEU) by the patient based on the EEG data.

26. The system of claim 24, wherein the BEA to the patient is proportional to a difference between a predefined initial brain energy (BEI) of the patient and a brain energy used (BEU) by the patient based on the EEG data.

27. The system of claim 24, wherein a change in cognitive activity done by the patient comprises a cognitive activity selected from a group consisting of meditation, sleep, rest in a peaceful environment, listen to relaxing music, guided stress reduction, guided heart rate biofeedback, guided neurofeedback biofeedback, relaxing hobby, gentle yoga, breathing exercises designed to help one relax, physical exercise, playing a game, having a conversation, walking a dog and any combination thereof.
